(19) United States
(12) Patent Application Publication
(10) Pub. No.: US 2007/0202085 A1
(43) Pub. Date: Aug. 30, 2007

(54) Systems and methods for monitoring immune responses and predicting outcomes in transplant recipients

(75) Inventors: Huaizhong Hu, Madison, WI (US); Stuart Knechtle, Fitchburg, WI (US); Jean Kwun, Madison, WI (US)

Correspondence Address: Medlen & Carroll, LLP, Suite 350, 101 Howard Street, San Francisco, CA, 94105, US

(73) Assignee: Renovar Incorporated, Madison, WI (US)

(21) Appl. No.: 11/653,743

(22) Filed: Jan. 16, 2007

Related U.S. Application Data
(60) Provisional application No. 60/759,254, filed on Jan. 13, 2006.

(51) Int. Cl. A61K 35/12; C12Q 1/68; G01N 33/53

(52) U.S. Cl. 424/93.7; 435/6; 435/7.92

(57) ABSTRACT

The present invention is related to transplant rejection. In particular, the present invention relates to determining the functional status of alloreactive T cells and correlating the functional status to in vivo immune responses (e.g., tolerance, rejection, or absence of rejection mediated by T cells). The present invention finds use in basic research, clinical (e.g., transplant) and therapeutic settings.

This application claims priority to U.S. Provisional Patent App. No. 60/759,254, filed Jan. 13, 2006, the entire contents of which are hereby incorporated by reference.

The present invention was funded, in part, under NIH grant RO1 AI050938-03. The government may have certain rights in the invention.

### FIELD OF THE INVENTION

The present invention is related to transplant rejection. In particular, the present invention relates to determining the functional status of lymphocytes (e.g., alloreactive T cells) within a graft recipient and correlating the functional status to in vivo immune responses (e.g., tolerance, rejection, or absence of rejection mediated by T cells). The present invention finds use in basic research, clinical (e.g., transplant) and therapeutic settings.

### BACKGROUND OF THE INVENTION

T cells play a central role in the rejection and acceptance of allogeneic organ transplants. Naive alloreactive T cells become activated when they are stimulated by alloantigens presented by professional antigen presenting cells (APC). While the majority of the activated T cells develop into effector cells to reject the transplant, a portion of these activated T cells evolve into memory cells. The memory T cells can mount a response to specific antigen stimulation more quickly than the naive T cells (See, e.g., Opferman et al., Science 1999:283: 1745-1748; Chalasani et al., Proc Natl Acad Sci U S A 2002:99: 6175-6180). In organ transplantation, memory T cells mediate accelerated rejection (See, e.g., Heeger et al., J Immunol 1999:163: 2267-2275; Deacock and Lechler, Transplantation 1992:54: 338-343; van Besouw et al., Transplantation 2000:70: 136-143). Under some circumstances, alloreactive T cells can become tolerant to alloantigen stimulation. In this case, the allogeneic transplant is accepted without the need for immunosuppression. Recipients of organ transplants are currently treated with continual long-term immunosuppressive therapy.

Long-term immunosuppressive therapy post transplantation typically involves the use of immunosuppressive agents such as cyclosporin A (CsA), rapamycin, FK506, corticosteriods, and antibodies to the interleukin (IL)-2 receptor. These drugs are typically taken over a long period of time, result in the global depletion of lymphocytes, and increase the risk of serious infection, nephrotoxicity, and cancer. Furthermore, some patients cannot tolerate doses of immunosuppressive agents that are sufficient to inhibit transplant rejection.

Immunosuppressive therapy may be reduced or discontinued if the patient develops immune tolerance to the graft (See, e.g., Calne et al., Lancet 1998:351: 1701-1702; Starzl et al., Lancet 2003:361: 1502-1510; Buhler et al., Transplantation 2002:74: 1405-1409; Knechtle mmunol Rev 2003:196: 237-246). However, no reliable parameter currently exists that fully indicates the functional status of alloreactive T cells of the recipient. Thus, there has been great difficulty identifying a tolerant recipient.

What is needed is a reliable and accurate method of identifying a tolerant recipient. For example, it would be of great value in clinical settings (e.g., transplant settings) as well as in basic research to have a method to characterize the functional status of alloreactive T cells of a graft recipient.

### SUMMARY OF THE INVENTION

The present invention is related to transplant rejection. In particular, the present invention relates to determining the functional status of lymphocytes (e.g., alloreactive T cells) within a graft recipient and correlating the functional status to in vivo immune responses (e.g., tolerance, rejection, or absence of rejection mediated by T cells). The present invention finds use in basic research, clinical (e.g., transplant) and therapeutic settings.

Accordingly, in some embodiments, the present invention provides a method for determining the likelihood of transplant rejection in a transplant recipient, comprising providing a sample from a transplant recipient; wherein the sample comprises T cells; exposing the sample to stimulator cells; measuring the level of one or more cytokines expressed by the T cells as a function of time; and correlating cytokine expression as a function of time with the likelihood of transplant rejection. In some embodiments, the stimulator cells comprise syngeneic antigen presenting cells. In some embodiments, the stimulator cells comprise allogeneic antigen presenting cells. In some embodiments, the stimulator cells comprise antigen presenting cells from the transplant donor. The present invention is not limited by the type of cytokine measured. Indeed, measurement of the expression of a variety of cytokines find use in the present invention including, but not limited to, IFN-γ, IL-2, IL-4, IL-5, IL-6, IL-12, TNF-α, and other cytokines. In some embodiments, the one or more cytokines expressed by the T cells are measured every 24 hours, however, the present invention is not so limited. For example, cytokine expression may be measured every 4, 6, 8, 12, 16, 18, 36, 48, 3 days, 6 days, 10 days, 20 days, 30 days or more over a period of days (e.g., 1-7 days or more), weeks (e.g., 1-4 weeks or more) or months (e.g., 1-6 months or more). In some embodiments, the one or more cytokines expressed by the T cells are measured for three or more days. In some embodiments, the method identifies a patient that has been tolerized to a transplanted graft. In some embodiments, the method discriminates between a naive and memory T cell response in said transplant recipient. In some embodiments, the patient is receiving one or more immunosuppressive drugs. The present invention is not limited by the type of immunosuppressive drug received by a patient. In some embodiments, the measuring occurs prior to transplantation. In some embodiments, the measuring occurs subsequent to transplantation. The present invention is not limited by the type of transplant recipient monitored utilizing a method of the present invention. Indeed, a variety of transplant recipients may find use of the present invention including, but not limited to, a bone marrow transplant recipient, an organ transplant recipient, a tissue transplant recipient and a skin transplant recipient. In some embodiments, measuring the level of one or more cytokines comprises detecting nucleic acid sequence. In some embodiments, measuring the level of one or more cytokines comprises detecting protein. The present invention is not limited by the method used to detect protein. Indeed, a variety of methods are contemplated to be useful for measuring cytokine protein including, but not limited to, enzyme linked immunosorbent assay (ELISA). In some embodiments, the present invention provides compositions and/or kits for carrying out methods and systems of the present invention (e.g., for use in clinical (e.g., transplant), therapeutic and/or research settings).

### DESCRIPTION OF THE DRAWINGS

FIG. 1 shows mouse skin graft survival. (A). C57BL/6J (H-2b) recipient mice were transplanted with skin derived from C57BL/6J (Syngeneic; n=6; filled circles) or BALB/c (H-2d) mouse with no treatment (n=12; filled triangles), treatment with cyclosporine (CsA) (n=12; filled squares) or a combination of CTLA4-Ig, anti-CD40L monoclonal antibody (mAb) and anti-CD25 mAb (n=6; open circles). Grafts were monitored once daily by visual inspection. Animals receiving syngeneic skin grafts had no rejection or complications. Animals receiving allogeneic skin grafts without treatment rejected the transplant with a mean survival time (MST) of 9 days. In mice treated with daily CsA (20 mg/kg), all transplants were rejected with the MST of 14 days, significantly longer (p<0.05) than the non-treated animals. The 6 mice that were treated on days 0, 2, 4 and 6 with combined 500 μg of CTLA-4Ig, 500 μg of anti-CD40L mAb and 250 μg of anti-CD25 mAb had prolonged graft survival with MST of 32.5 days, significantly longer than both nontreated (P<0.01) and CsA treated animals (P<0.05). Among the 6 mice, 3 accepted the graft without rejection during the observation period. (B). Representative mouse skin grafts. A syngeneic graft in a mouse was accepted without any complications (a,b). An allogeneic graft in a mouse without treatment was rejected with numerous infiltrating cells in the graft (c,d). An allogeneic graft in a mouse treated with CsA as rejected with infiltrating cells in the graft as well (e,f). An allogeneic graft in a mouse was accepted and shown to be healthy by visual inspection and histology 40 days after transplantation (g,h). This mouse was treated with costimulation blockade and anti-CD25 mAb.

FIG. 2 shows that IFN-γ kinetics differentiates the functional status of allogeneic T cells. Spleen cells derived from C57BL/6J (H-2b) mice that received a skin graft from Balb/c (H-2d) mice were challenged by irradiated spleen cells obtained from Balb/c (donor, filled squares), C3H (H-2k; third party, filled inverted triangle) and C57BL/6J mouse (autologous, filled triangle). Each combination was performed in 15 replicates. Cells were incubated at 37° C. in a humidified atmosphere containing 5% CO2. Starting from day 1 (1 day after the initiation of the culture), 150 μl of culture supernatant was harvested from each well, and 3 wells per day until day 5. Concentration of IFN-γ in the culture supernatant was determined by ELISA. (a). a representative non-transplanted naive mouse; (b), a representative rejecting mouse; (c), a representative experiment using spleen cells obtained from a mouse 100 days after the graft rejection. (d), a representative mouse that accepted the allogeneic skin graft with the combination therapy comprising the costimulation blockade and anti-CD25 mAb. (e), a representative mouse that rejected the allogeneic graft with CsA treatment. (f), a representative skin-grafted mouse treated with CsA; this mouse was sacrificed on day 7 after transplantation while the graft was not rejected yet. Each data point represents the mean±standard deviation.

FIG. 3 shows IFN-γ expression detected by ELISPOT assay. Spleen cells derived from C57BL/6J (H-2b) mice that received a skin graft from Balb/c (H-2d) mice were challenged by irradiated spleen cells obtained from Balb/c (donor), C3H (H-2k, third party) and C57BL/6J mouse (autologous) for 48 hours before the detection of WN-γ expression. (A). a, a representative non-transplanted naïve mouse; b, a representative rejecting mouse; c, a representative experiment using spleen cells obtained from a mouse 100 days after the graft rejection. d, a representative mouse that accepted the allogeneic skin graft with the combination therapy. (B). Experiments were done as described in A. Rejecting mice had significantly higher numbers of spots than the naive and tolerant mice (P<0.05).

FIG. 4 depicts the determination of T cell proliferation by CFSE staining and flow cytometry. (A). Responder cells (4×10⁵) were stained by CFSE and were stimulated by 4×10⁵ irradiated Balb/c mouse cells. After 4 days culture, cells were stained by monoclonal antibody directed separately at CD3, CD4 and CD8 before FACS analysis. Responder cells were derived separately from naive C57BL/6J mice, or C57BL/6J mice that were rejecting or accepted a Balb/c skin graft. (B). Experiments were conducted as described in A. Percentage of proliferating cells was calculated by proliferating cells (CFSE_{low} cells)/proliferating cells (CFSE_{low} cells)+nonproliferating cells (CFSE_{high} cells). Each bar represents mean (±SE). Both naive and rejection mice had a significantly higher (P<0.05) T cell proliferation than the tolerant mice.

FIG. 5 shows IFN-γ producing cells in naive and rejected mice after PMA and lonomycin activation. (A). IFN-γ production by NK, NKT and T cells. Isolated fresh splenocytes from naive and skin graft rejection mice were activated with PMA (10 ng/ml) and lonomycin (4 μg/ml) for 4 hrs and cultured with Golgi-stop (Brefeldin A) for another 4 hours. Cells were then harvested for staining with anti-CD3 mAb-FITC, anti-NK1.1 mAb-APC before the permeabilization for intracellular staining with PE-coupled mAbs directed at IFN-γ. Data shown represents the mean±SE of three experiments. (B). Kinetics of IFN-γ producing T cell after mixed lymphocyte reaction (MLR). Isolated fresh splenocytes from naive and skin graft rejecting C57BL/6J mice were separately stimulated by irradiated Balb/c donor mouse spleen cells. Cultured cells were collected at 24, 48 and 72 hours after MLR and processed as described in A for FACS analysis. (C). Kinetics of IFN-γ producing T cell after mixed lymphocyte reaction (MLR). Experiments were conducted as described in B. Data shown are Mean±SE.

FIG. 6 shows the Kinetics of Leukocyte Repopulation in the Peripheral Blood of CAMPATH Patients. (A) T cell (CD3), B cell (CD20), and monocyte (CD14) numbers were averaged for CAMPATH-treated allograft patients and are shown as percent baseline. 29 patients are included in the averages up to month 12, 24 patients are included for month 24, and 6 patients for month 36. (B) Absolute cell counts of T cell subsets after CAMPATH induction. Cell numbers are averaged as in (A). (C) Absolute lymphocyte counts for CAMPATH versus control patients at pre-transplant and month 12 post-transplant time-points. Shown are error bars for standard error of the mean.

FIG. 7 shows CFSE-MLR Dot Plot Analysis for CAMPATH-Treated Patient UW19. Proliferation of CD3+, CD4+, and CD8+ lymphocyte populations in response to donor and third party MHC are measured by loss of CFSE intensity. CFSE and PE were analyzed on FL1 and FL2 channels, respectively.

FIG. 8 shows CFSE-MLR Proliferation Analysis. Scatter plot of the percent proliferation (% CFSE-low) of CD3+, CD4+, and CD8+ cells for CAMPATH (circles) versus control patients (squares). (filled circle) CAMPATH patient response to donor Ag; (unfilled circle) CAMPATH patient response to third party Ag; (filled square) control patient response to donor Ag; (unfilled square) control patient response to third party Ag. Bars depict average proliferation for all patients in that group.

FIG. 9. Cytokine Kinetics for IFN-γ. MLRs were set up in quintuplet wells and supernatants were taken at 24-hour intervals for 5 days. IFN-γ levels (pg/ml) were subsequently measured by multiplex fluorescent bead detection. (filled square) response to donor antigen, (filled triangle) response to third party antigen, (filled circle) response to autologous antigen.

### DEFINITIONS

To facilitate an understanding of the present invention, a number of terms and phrases are defined below:

As used herein, the term “syngeneic” refers to genetically identical or closely related organisms, cells, tissues, organs, and the like.

As used herein, a “syngeneic skin graft” refers to a skin graft wherein the host for the skin graft and the source of the skin graft are individuals that are genetically identical or sufficiently closely related such that the graft and the host do not interact antigeneically.

As used herein, the term “allogeneic” refers to organisms, cells, tissues, organs, and the like from, or derived from, individuals of the same species, but wherein the organisms, cells, tissues, organs, and the like are genetically different one from another (e.g., have one, two or more MHC antigen mismatches).

As used herein, the term “xenograft” refers to a transplant in which the donor and recipient are of different species.

As used herein, an “allogeneic skin graft” refers to a skin graft wherein the host for the skin graft and the source of the skin graft are individuals of the same species that are sufficiently unlike genetically such that the graft and the host are likely to interact antigeneically. An allogeneic transplant may be rejected over time in the absence of an intervention (e.g., administration of an immunosuppressive agent (e.g., CsA)) to inhibit transplant rejection (e.g., caused by alloreactive T cells).

As used herein, the term “transplant rejection” refers to a partial or complete destruction (e.g., functional and/or structural) of a transplanted cell, tissue, organ, or the like on or in a recipient of said transplant (e.g., due to an immune response generated by the recipient).

As used herein, the term “tolerance” refers to the absence of transplant rejection (e.g., the absence of a recipient immune response to the transplanted graft). “Peripheral tolerance” refers generally to tolerance acquired by mature lymphocytes in peripheral tissues.

As used herein, the term “host” refers to an organism (preferably the organism is a mammal), a tissue, organ, or the like that is the recipient of a transplanted cell, tissue, organ, or the like. The terms “host” and “recipient”, when referring to transplant hosts or recipients are used interchangeably, unless indicated otherwise herein.

As used herein, the term “isolated” when used in relation to material (e.g., a cell) refers to a material that is identified and separated from at least one component or contaminant with which it is ordinarily associated in its natural source. An isolated material is such present in a form or setting that is different from that in which it is found in nature.

As used herein, the term “purified” or “to purify” refers to the removal of components (e.g., contaminants) from a sample.

As used herein, the term “transplantation” refers to the process of taking a cell, tissue, organ, or the like, herein called a “transplant” or “graft” from one subject and placing the transplant into a (usually) different subject. The subject that provides the transplant is called the “donor” and the subject that receives the transplant is called the “host” or “recipient”. Typically, the host (i.e., the recipient of the transplant or graft; referred to herein as “graft recipient” or “transplant recipient”) is a mammal, such as a human. The transplant can include any transplantable cell, tissue, organ or the like. For example, it can include a kidney, liver, heart, lung, bone marrow, skin, etc. Thus, a graft wherein the donor and host are genetically identical is a syngeneic graft. Where the donor and host are the same subject, the graft is called an autograft. The invention relates to all types of grafts.

As used herein, the terms “immunoglobulin” and “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like (e.g., that is to be the recipient of a particular treatment (e.g., transplant graft) or that is a donor of a graft. The terms “subject” and “patient” are used interchangeably in reference to a human subject, unless indicated otherwise herein (e.g., wherein a subject is a graft donor).

As used herein, the term “cytokine kinetic assay” (e.g., an “INF-γ kinetic assay”) refers to detecting the expression and/or level of cytokine (e.g., IFN-γ) expressed by T cells (e.g., within a sample (e.g., isolated from a graft recipient) over a period of time when incubated with other cells (e.g., allogenic, syngeneic, or third party splenocytes). Cytokine (IFN-γ) expression and/or levels can be monitored one or more times daily for one, two, three, four, five or more days. In some preferred embodiment, the kinetics assay measures the number of cells (e.g., T cells) that express and/or secrete a cytokine (e.g., IFN-γ) over a period of time. Thus, in preferred embodiments, a cytokine kinetic assay is capable of discriminating between a primary (e.g., naive) and a secondary (e.g., an effector or memory) T cell response (e.g., based upon the time of expression/secretion of the cytokine rather than upon the amount of cytokine expression/secretion).

As used herein, the term “antigen presenting cells” refers to cells that are able to present antigens to T cells (e.g., for stimulation and activation of the T cells). Such cells include, but are not limited to, macrophages, dendritic cells and B cells.

As used herein, the term “predicting transplant rejection risk in a subject” refers to determining the risk of a subject rejecting a transplant (e.g., graft tissue, cell, organ or the like) at any point following the transplant. In some preferred embodiments, predicting transplant rejection risk is based on characterizing lymphocytes (e.g., detecting and/or characterizing a dynamic T cell response) of a graft recipient utilizing an cytokine (e.g., IFN-γ) kinetic assay (e.g., capable of discriminating between a primary (e.g., naïve) and a secondary (e.g., an effector or memory) T cell response).

As used herein, the term “reagents for a cytokine kinetics assay” refers to reagents specific for (e.g., sufficient for) the detection of one or more cytokines (e.g., IFN-γ or GM-CSF), for example, in an ELISPOT assay. In some embodiments, the reagent is an antibody specific for a cytokine (e.g., IFN-γ). In some embodiments, the reagents further comprise additional reagents for performing detection assays, including, but not limited to, controls, buffers, etc.

As used herein, the term “determining a treatment course of action” as in “determining a treatment course of action based on said predicting transplant rejection risk” or “determining a treatment course of action based on said diagnosing transplant rejection,” refers to the choice of treatment administered to a subject (e.g., graft recipient). For example, if a subject is found to be at increased risk of graft rejection (e.g., of a cell, tissue, organ or the like) or to be undergoing graft rejection, anti-rejection therapy may be started, increased, or changed from one treatment type (e.g., pharmaceutical agent) to another. Conversely, if a subject is found to be at low risk for organ rejection, anti-rejection therapy may not be administered or levels of anti-rejection therapy (e.g., CsA or rapamycin) may be decreased. In some embodiments, the treatment course of action is “continued monitoring” in which no anti-rejection treatment is administered but the cytokine kinetics assay is continued (e.g., monitored regularly (e.g., using the methods of the present invention)).

As used herein, the term “determining the efficacy of said anti-rejection drug based on said detecting” refers to determining if an anti-rejection drug is preventing transplant rejection based on, for example, utilizing a cytokine (e.g., IFN-γ) kinetics assay of the present invention to characterize a transplant graft recipient subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “in vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., transplant rejection). Test compounds comprise both known and potential therapeutic compounds (e.g., known immunosuppressents including, but not limited, CsA and antilymphocyte drugs such as OKT3, as well as those whose immunosuppressive effects are yet to be determined (e.g., using systems and methods of the present invention). A test compound can be determined to be therapeutic by screening using the screening methods of the present invention.

As used herein, the term “sample” is used in its broadest sense. For example, in some embodiments, it is meant to include a specimen or culture (e.g., microbiological culture). In preferred embodiments, it is meant to include a biological sample.

The present invention is not limited by the type of biological sample used or analyzed. The present invention is useful with a variety of biological samples including, but are not limited to, tissue (e.g., organ (e.g., heart, liver, brain, lung, stomach, intestine, spleen, kidney, pancreas, and reproductive (e.g., ovaries) organs), glandular, skin, and muscle tissue), cell (e.g., blood cell (e.g., lymphocyte or erythrocyte), muscle cell, tumor cell, and skin cell), gas, bodily fluid (e.g., blood or portion thereof, serum, plasma, urine, semen, saliva, etc), or solid (e.g., stool) samples obtained from a human (e.g., adult, infant, or embryo) or animal (e.g., cattle, poultry, mouse, rat, dog, pig, cat, horse, and the like). Biological samples may be obtained from all of the various families of domestic animals, as well as feral or wild animals, including, but not limited to, such animals as ungulates, bear, fish, lagamorphs, rodents, etc.

Biological samples also include biopsies and tissue sections (e.g., biopsy or section of tumor, growth, rash, infection, or paraffin-embedded sections), medical or hospital samples (e.g., including, but not limited to, blood samples, saliva, buccal swab, cerebrospinal fluid, pleural fluid, milk, colostrum, lymph, sputum, vomitus, bile, semen, oocytes, cervical cells, amniotic fluid, urine, stool, hair and sweat), and laboratory samples (e.g., subcellular fractions).

### DETAILED DESCRIPTION OF THE INVENTION

T cells play a central role in the rejection and acceptance of allogeneic transplants (e.g, organ transplants). While the majority of the activated T cells develop into effector cells to reject the transplant, a portion of these activated T cells evolve into memory cells. Memory T cells can mount a response to specific antigen stimulation that is more robust and quicker than naive T cells. In organ transplantation, memory T cells mediate accelerated rejection (See, e.g., Heeger et al., J Immunol 1999:163: 2267-2275; Deacock and Lechler, Transplantation 1992:54: 338-343; van Besouw et al., Transplantation 2000:70: 136-143). Under some circumstances, alloreactive T cells can become tolerant to alloantigen stimulation. In this case, the allogeneic transplant is accepted without the need for continued immunosuppression. Recipients of organ transplants are currently treated with continual long-term immunosuppressive therapy. However, due to the lack of reliable parameters to indicate the functional status of alloreactive T cells within the recipient, it remains very difficult currently to identify tolerant recipients.

The diagnosis of acute rejection, chronic rejection, and polyoma viral nephritis is presently based on the histopathology of biopsy samples. Nevertheless, biopsy histology does not necessarily reflect the recipient T cell functional response to the graft (See, e.g., Solez et al., Kidney Int 1993:44: 411-422). In the past several decades, an enormous effort has been devoted to developing lab tests that can accurately report the recipient immune status towards donor alloantigens. The prototypical test is the mixed lymphocyte reaction (MLR). T cell responses to alloantigens are determined by cell proliferation, cytokine secretion, or intracellular ATP elevation. These methods evaluate the T cell response at a single time point, thereby lacking information regarding T cell functional response status as a function of time (See, e.g., Cartwright et al., Transpl Immunol 2000:8: 109-114).

It is well known that the primary and memory response of B cells to antigens is reflected by their antibody production kinetics. A memory response produces antibodies much earlier with higher titer and affinity than the primary response (See, e.g., Goldsby et al., in Immunology, Ed. Kuby New York: W.H. Freeman and Company, 2000:269-300). It is also known that primed T cells make a quicker response than naïve T cells when they are rechallenged by specific antigens (See, e.g., Opferman et al., Science 1999:283: 1745-1748). Thus, during the development of the present invention, it was determined whether the cytokine kinetics of T cells would reflect the T cell functional response (e.g., within a graft recipient) in a similar manner. To test this hypothesis, a mouse skin graft model was used to establish an in vitro cytokine kinetics assay (e.g., an INF-γ kinetics assay (See, e.g., Example 1, below)). Although a number of cytokines can be used for a cytokine kinetics assay of the present invention (e.g., IFN-γ and GM-CSF), IFN-γ is a preferred cytokine on the basis of previous studies that indicate the usefulness of IFN-γ in the detection of primed T cells by enzyme-linked immunospot (ELISPOT) assay and flow cytometry. Accordingly, the present invention provides a cytokine kinetics assay (e.g., IFN-γ kinetics assay) that finds use in the characterization of the functional status and/or response of lymphocytes present within a graft recipient (e.g., of a subject's alloreactive T cells; See, e.g., Examples 1 and 3, below).

The present invention provides that the dynamic portion of the T cell response is highly important (and informative) for determining the nature of the in vivo T cell response. For this reason, a cytokine kinetics assay reflects lymphocyte (e.g., T cell) functional status within a graft recipient. A good example of a kinetics characteristics is the differentiation of the primary and secondary antibody responses by B lymphocytes: for example, in secondary responses the antibody is produced 3-4 days earlier with a higher titer and affinity.

Using a cytokine kinetics assay of the present invention (e.g., IFN-γ kinetics assay), the present invention demonstrates the similarity between T and B cell responses (e.g., T cell kinetic responses within a graft recipient). For example, experiments conducted during the development of the present invention that utilized a cytokine kinetics assay of the present invention demonstrated that naive T cells respond to specific alloantigen stimulation at least 2 days later than the effector/memory T cells, and that effector cells mount a response 1 day earlier than the memory cells alone (See Examples 1-4, below). Based on the levels of IFN-γ in the culture supernatant, the effector and memory cells mount a much stronger response than the naïve T cells (however, in some embodiments, as described herein, the amount of cytokine (e.g., IFN-γ) appears less important than the time at which the cytokine is expressed/secreted).

Furthermore, the cytokine kinetics assay of the present invention is able to characterize the functional status of lymphocytes in a graft recipient that has or has not received immunosuppressive agents (e.g., rapamycin, cyclosporine (CsA), etc.). In particularly preferred embodiments, the present invention provides a cytokine (e.g., IFN-γ) kinetics assay that is capable of determining the likelihood of a transplanted graft being rejected. In the same manner, the present invention provides a method (e.g., utilizing a IFN-γ kinetics assay) of determining whether a graft recipient has, or will, become tolerized to a graft.

For example, the effect of CsA immunosuppression in mouse skin transplantation was reflected by the IFN-γ kinetics assay (See Examples 1-5 below). CsA is not able to prevent the rejection of skin transplants in the mouse, and CsA is ineffective in inhibiting effector/memory cells while it can prevent naive T cell activation. In mice treated with CsA, there was no response to third-party or autologous cells, but the EFN-γ kinetics assay showed an effector/memory pattern with donor cell stimulation. These results revealed 1) that the recipient T cells were suppressed, since no response was made to third-party stimulation, and 2) that the recipients had effector/memory cells towards the donor alloantigens that were not inhibited completely by the immunosuppressive agent. The effector cells were responsible for rejecting the skin graft.

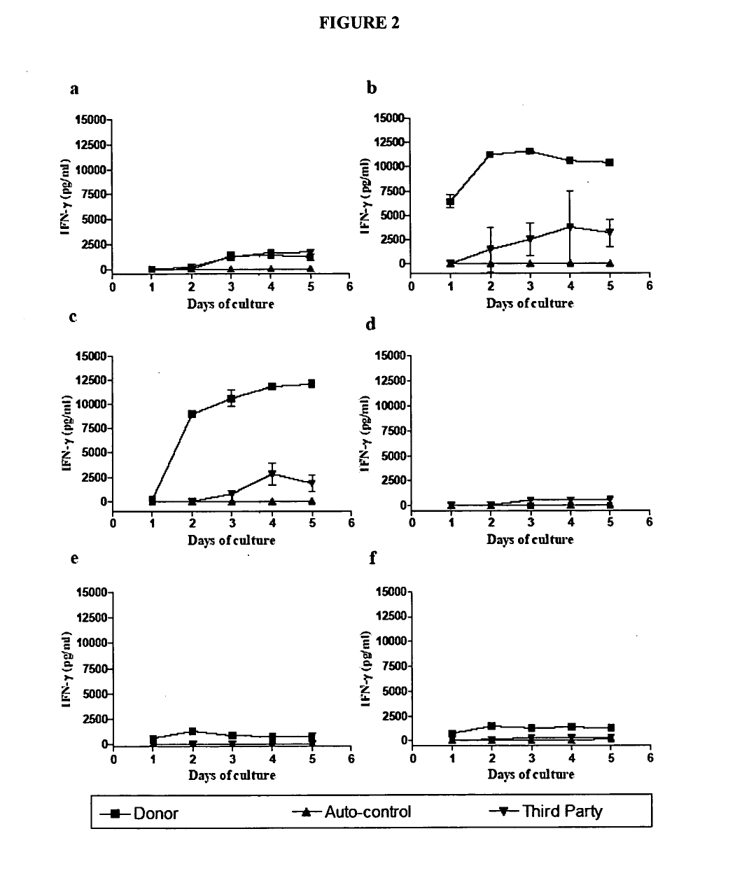

As shown in FIG. 2, the IFN-γ levels were not very high in the effector/memory response compared to those mice with graft rejection that were not treated with CsA. Nevertheless, the secretion of IFN-γ started on day 1 after the culture and increased to a higher level on the following days, a typical pattern of an effector/memory response. Therefore, the kinetics assay of the present invention is able to differentiate T cell functional states in this situation. Thus, in some preferred embodiments, and in contrast to heretofore utilized methods of characterizing transplant status, it is the kinetic pattern (e.g., of cytokine (e.g., IFN-γ)) of expression and/or secretion, rather than the level of IFN-γ, that characterizes the T cell functional status in a graft recipient. However, in some embodiments, the level of cytokine expressed and/or secreted is also used (e.g., together with kinetic data) to characterize T cell functional status in a graft recipient.

Additionally, donor-specific tolerance is also unveiled by a cytokine (e.g., IFN-γ) kinetics assay of the present invention. In previous studies, it was shown that treatment with anti-CD40L mAb and CTLA-4Ig could prolong the allograft survival in mouse skin transplantation (See, e.g., Kirk et al., Proc Natl Acad Sci U S A 1997:94: 8789-8794; Larsen et al., Nature 1996:381: 434-438). Nevertheless, certain strains of mouse are relatively resistant to the effect of this costimulation blockade (See, e.g., Trambley et al., J Clin Invest 1999:104:1715-1722; Guo et al., Transplantation 2001:71: 1351-1354). A recent report demonstrated that blockade of IL-2 pathway in addition to costimulation significantly extended allograft survival in mouse skin transplantation (See, e.g., Jones et al., J Immunol 2002:168: 1123-1130). In mice treated with costimulation and anti-CD25 blockade, the graft was accepted without further immunosuppression. Cytokine kinetic assays of the present invention demonstrated that lymphocytes from these tolerant mice showed a naive response to the third-party cells that failed to secrete detectable IFN-γ in the presence of donor cells. Thus, based on the above-described correlation between the skin graft outcome and the IFN-γ kinetics assay, methods of the present invention are capable of revealing the T cell functional state in multiple situations. Thus, the present invention provides a method of determining donor-specific tolerance in a graft recipient comprising utilizing a cytokine (e.g., IFN-γ) kinetics assay. For example, in some preferred embodiments, absence of detectable cytokine (e.g., IFN-γ) secreted by lymphocytes isolated from a graft recipient as measured by a cytokine (e.g., IFN-γ) kinetics assay (e.g., characterizing the response as a naive response and not a memory response) can be utilized to indicate donor-specific tolerance in a graft recipient.

Alloreactive effector/memory T cells contribute to both acute and chronic allograft rejection (See, e.g., Heeger et al., J Immunol 1999:163: 2267-2275; Najafian et al., J Am Soc Nephrol 2002:13: 252-259). Memory T cells induce accelerated graft rejection. Alloreactive memory T cells are generated not only in transplant recipients, but also in individuals who have never been exposed to foreign tissues (See, e.g., Heeger et al., J Immunol 1999:163: 2267-2275). It is proposed that in the latter case alloreactivity in the T cell memory pool is caused largely by cross-reactivity; in other words, exposure to viral and bacterial antigens over time leads to the development of memory T cells that also recognize alloantigens (See, e.g., Turgeon et al., J Surg Res 2000:93: 63-69; Williams et al., J Immunol 2001:167: 4987-4995; Pantenburg et al., J Immunol 2002:169: 3686-3693; Brehm et al., J Immunol 2003:170: 4077-4086). Therefore, differentiating the functional state of alloreactive T cells in transplant recipients may be important both before and after transplantation. Accordingly, in some embodiments, a cytokine (e.g., IFN-γ) kinetics assay is utilized to characterize the functional state of alloreactive T cells in transplant recipients both prior to as well as after transplantation.

The cytokine kinetics assay of the present invention is able to characterize the influence of effector/memory T cells on the rejection of the skin graft, even in the presence of immunosuppression. Skin-grafted mice were treated with two different immunosuppressive protocols, and two different outcomes were observed. Mice treated with CsA failed to accept the graft. In these mice, effector/memory T cells were detected, and CsA did not suppress this T cell population. Of the six mice that were treated with costimulation and anti-CD25 blockade, three had graft rejection and three accepted the graft. In the rejecting mice, the IFN-γ kinetics assay showed an effector/memory response, indicating that the treatment failed to induce donor specific tolerance. However, in mice that accepted the graft, the IFN-γ kinetics assay revealed no response to the donor antigen stimulation while maintaining a naïve response to the third-party cells, a clear picture of donor-specific tolerance. Thus, in some embodiments, the present invention provides methods of characterizing memory T cells in a graft recipient. The present invention provides support for the proposal by Lakkis and Sayegh that memory T cells are indeed a hurdle to immunologic tolerance (See, e.g., Lakkis and Sayegh, J Am Soc Nephrol 2003:14: 2402-2410).

The present invention further provides a method, using a cytokine kinetics assay, to characterize naive and memory T cells by their phenotypic features (e.g., CD4+ and CD8+ T cell subsets). Recent studies propose the existence of two subsets of CD4+ and CD8+ memory T cells based on the expression of the homing receptors CC-chemokine receptor 7 (CCR7) and L-selectin (CD62L). The CCR7+CD62Lₕᵢ “central” memory T cells (T_{CM}) and CCR7- CD62Lₗₒ “effector” memory T cells (T_{EM}) have distinct migratory and functional characteristics (See, e.g., Sallusto et al., Nature 1999:401: 708-712; Weninger et al., J Exp Med 2001:194: 953-966; Iezzi et al., J Exp Med 2001:193: 987-993). T_{CM} circulate through secondary lymphoid tissues and produce IL-2 but little IFN-γ and no perforin. T_{EM} circulate through nonlymphoid peripheral tissues and produce IFN-γ, perforin, and IL-4 but little IL-2. It has been proposed that the secondary lymphoid tissue resident T_{CM} are responsible for replenishing the memory T cell pool because of their increased proliferative capacity, whereas the nonlymphoid tissue resident T_{EM} mediate effector functions and rapid elimination of antigen (See, e.g., Sallusto et al., Nature 1999:401: 708-712; Reinhardt et al.,. Nature 2001:410: 101-105; Masopust et al., Science 2001:291: 2413-2417). Thus, in some embodiments, the present invention combines the analysis of these T cell phenotypical features with a cytokine (e.g., IFN-γ) kinetics assay of the present invention.

CAMPATH-1H (alemtuzumab) is a humanized monoclonal antibody that binds the cell surface glycoprotein CD52. In peripheral blood, the antibody targets the CD52 antigen expressed on T and B lymphocytes, NK cells, monocytes, and dendritic cells, inducing their rapid, transient depletion both in vitro and in vivo (See, e.g., Buggins et al., Blood 2002; 100(5): 1715; Ratzinger et al., Blood 2003; 101(4): 1422; Riechmann et al., Nature 1988; 332(6162): 323). CAMPATH has been used as a therapeutic strategy for a wide range of immune-mediated diseases including multiple sclerosis, rheumatoid arthritis, and chronic lymphocytic leukemia (See, e.g., Moreau et al., Mult. Scler. 1996; 1(6): 357; Confavreux and Vukusic, Clin. Neurol. Neurosurg. 2004; 106(3): 263; Cohen and Nagler, Autoimmun. Rev. 2004; 3(2): 21; Hale et al., Bone Marrow Transplantation 2002; 30(12): 797). It is also a well-established treatment for the prevention of graft versus host disease (GVHD) in bone marrow transplantation (See, e.g., Hale et al., Bone Marrow Transplantation 2002; 30(12): 797). More recently, CAMPATH-1H has been applied to solid organ transplants, including kidney, pancreas, and intestine (See, e.g., Calne et al., Nippon Geka Gakkai Zasshi 2000; 101(3): 301; Knechtle et al., Am. J. Transplant 2003; 3(6):722; Garcia et al., Transplantation Proceedings 2004; 36(2): 323; Kandaswamy et al., American Journal of Transplantation 2004; 4: 536).

In renal transplant recipients, CAMPATH-1H induction therapy has allowed allografts to be maintained with reduced immunosuppression. Calne et al. reported a low rejection rate over a 2-year period in 31 patients receiving CAMPATH perioperatively, with subsequent low dose cyclosporine monotherapy, a condition described as ‘almost’ or prope tolerance (See, e.g., Calne et al., Nippon Geka Gakkai Zasshi 2000; 101(3): 301). Subsequently, a pilot study demonstrated that a majority of renal allograft recipients treated with CAMPATH induction therapy maintained good graft function while on low dose rapamycin monotherapy (See, e.g., Knechtle et al., Am. J. Transplant 2003; 3(6):722). Kirk et al. reported a clinical trial in which CAMPATH-1H induction therapy was used in renal transplant patients without any other immunosuppression (See, e.g., Kirk et al., Transplantation 2003; 76(1): 120). Despite early rejection episodes that may likely have been mediated by monocytes, rejection was reversible, and patients could likewise be maintained on rapamycin monotherapy. More recently, the experience with CAMPATH-1H for kidney transplantation at the University of Wisconsin-Madison has been associated with a significant reduction-in rejection rates, and improved graft survival for patients with delayed graft function (See, e.g., Knechtle et al., Surgery 2004; 136(4): 754).

Undertaking mechanistic studies for CAMPATH-mediated immunodepletion is an important step in defining the benefits of this antibody in solid organ transplant. Therefore, in an effort to understand the characteristics of repopulating lymphocytes in CAMPATH-treated renal allograft patients, the responsiveness of graft recipient T cells to donor antigen in vitro was analyzed using methods of the present invention. Using methods of the present invention (e.g., cytokine (e.g., IFN-γ) kinetics assays), it was observed that the degree of T cell responsiveness to third party antigen is greater in the CAMPATH-treated group compared to those treated with anti-CD25 antibody, indicating that CAMPATH-1H/rapamycin does not over-immunosuppress, but allows repopulating T cells to maintain responses to foreign antigen. Furthermore, a number of CAMPATH-treated patients are completely unresponsive to donor antigen via the direct pathway of allorecognition. Thus, the present invention provides a method for determining the degree of T cell responsiveness within a graft recipient that has received one or more immunosuppressive agents (e.g., drugs) to third party antigen comprising utilizing a IFN-γ kinetics assay. Such information is useful for determining course of treatment or alteration of treatments in transplant subjects.

MLR data demonstrate that at one year post-transplant, repopulated T cells of CAMPATH/rapamycin-treated patients are able to respond to third party alloantigen quite well. As such, these patients can likely respond capably to foreign antigens. Clinical data support this finding, as CAMPATH-1H induction therapy does not render patients more susceptible to viral or bacterial infections compared to conventional triple immunosuppression (See, e.g., Knechtle et al., Am. J. Transplant 2003; 3(6):722). This is despite the fact that T cell numbers are low and CD4⁺/CD8⁺ T cell ratios are decreased, thereby limiting the effect of T helper cells.

Cytokine kinetic assays of the present invention demonstrated that the average responses to donor alloantigen did not differ significantly between CAMPATH-1H/rapamycin and anti-CD25/CsA/MMF/Pred groups, suggesting that the two regimens could be equally effective immunosuppressive therapies. However, when looking individually at each patients T cell proliferation profile, 4 of 15 CAMPATH versus 0 of 8 control patients were completely unresponsive to donor alloantigen as determined by CFSE-MLR and IFN-γ kinetics. Thus, in some embodiments, the present invention provides that T cell tolerance to intact alloantigen has been established in at least a subset of CAMPATH/rapamycin-treated patients.

CAMPATH/rapamycin may act by promoting the generation of CD4⁺CD25⁺ T regulatory cells (Tregs). Lechler et al. have shown that in renal allograft recipients treated by more conventional therapies, CD4⁺CD25⁺ Treg's do not play a significant role in suppressing alloresponses (See, e.g., Game et al., J. Am. Soc. Nephrol. 2003; 14(6): 1652). However, Rudensky et al. have shown that CD4⁺CD25⁺ Treg's can homeostatically proliferate in lymphopenic mice (See, e.g., Gavin et al., Nat. Immunol. 2002; 3(1): 33). It has also recently been shown that unlike cyclosporine A, rapamycin does not inhibit the expression of FoxP3 (See, e.g., Baan et al., Transplantation 2005; 80(1): 110). Therefore, the possibility exists that Treg's expand in CAMPATH-1H/rapamycin treated patients, efficiently suppressing alloresponses upon repopulation. Alternatively, treatment with CAMPATH may be a conduit for the expansion or increased effectiveness of CD8⁺CD28-FOXP3⁺ T suppressor cells (Ts) (See, e.g., Liu et al., International Immunology 1998; 10(6): 775). This Ts cell population has been shown to inhibit both CD4⁺ and CD8⁺ effector T cells specific for the direct pathway of allorecognition (See, e.g., Liu et al., International Immunology 1998; 10(6): 775). Therefore, CD8⁺CD28-FOXP3⁺ Ts cells could also play a role in increased hyporesponsiveness of T effector cells that recognize intact alloantigen in CAMPATH-treated patients.

The clinical outcomes of the CAMPATH and control groups did not differ significantly at month 12 post-transplant. However, 10 CAMPATH patients were on single drug maintenance therapy. Eight of these 10 were hypo- or unresponsive to donor antigen as measured by IFN-γ kinetics. Five of 8 control patients who were on triple maintenance therapy were likewise hypo-responsive to donor antigen. These data suggest that CAMPATH-1H-treated patients on long-term monotherapy have an equal chance of being hyporesponsive to donor antigen as those on triple therapy, providing a mechanistic foundation for the beneficial use of CAMPATH-1H.

Thus, the present invention provides a method of monitoring immune responses in a patient who is being evaluated as a transplant (e.g., organ) recipient and/or is receiving at least one immunosuppressive drug. This method includes the steps of analyzing the immunological responses of a set or subset of lymphocytes from a sample (e.g., blood or tissue sample) by determining at least one level of functional activity using a cytokine (e.g., IFN-γ) kinetics assay and comparing it with the immunological responses of appropriate controls (e.g., exposure to syngeneic or third party lymphocytes). The immune status assessment of a subject can then be based on this comparison.

In some embodiments, the subject is a transplant recipient. For example, the subject may be a recipient of an organ (e.g., heart, lungs, kidney, pancreas, liver, small bowel or other organs), tissues, skin, or bone marrow transplant. A subject may be administered one or more immunosuppressive drugs including, but not limited to, calcineurin inhibitors, enzyme inhibitors, antimetabolites, lymphocyte depleting drugs, corticosteroids, or other immune modulators. Drug combinations may also be administered. The overall effect of drugs on immune responses may be measured using a kinetic assays of the present invention that determine lymphocyte (e.g., T cell) functional status in a graft recipient.

One aspect of the invention is a method for predicting a clinical outcome in a patient (e.g., transplant patient or patient with autoimmune disease) who is receiving none or one or more immunosuppressive drugs. The method utilizes measured ranges of lymphocyte function status derived from a cohort of apparently healthy individuals as a means of defining normal ranges of reactivities, and includes the steps of determining at least one value of lymphocyte functional status in a sample (e.g., blood) from a patient before or after administration of immunosuppressive drug(s); determining whether the lymphocyte response of the cells from the patient receiving the immunosuppressant drug is higher or lower than the range defined for apparently healthy individuals, or falls within it; and providing a guide for therapy and predicting a clinical outcome based on the comparison. Clinical outcomes or conditions which may be predicted include transplant rejection, over-medication, and infection. For example, depending on parameters established, a lymphocyte response that is absent or that falls in a low range indicates high immunosuppression and may be indicative of over-medication which may lead to organ toxicity, infection, or, in the long term, cancer. A lymphocyte response that falls in a range of high T cell alloresponsiveness may be indicative of a low immunosuppressed condition, which may be indicative of infection or a course leading to organ rejection. Alternatively, a lymphocyte response that falls in a moderate range of alloresponsiveness may indicate that stability of the immunological response has been achieved and that no changes in therapeutic regimen are warranted at that time.

Another aspect of the invention is to use the assay to monitor patients who are being weaned from immunosuppressant drug(s), or for measuring patient compliance with medication prescriptive instructions.

In some embodiments, an automated detection assay is utilized to detect one or more cytokines. Methods for the automation of immunoassays include those described in U.S. Pat. Nos. 5,885,530, 4,981,785, 6,159,750, and 5,358,691, each of which is herein incorporated by reference. In other embodiments, the immunoassay described in U.S. Pat. Nos. 5,599,677 and 5,672,480; each of which is herein incorporated by reference, is utilized to detect one or more cytokines. In some embodiments, the analysis and presentation of results (e.g., cytokine expression as a function of time) is also automated. For example, in some embodiments, software that generates a prognosis (e.g., for graft survival and/or acceptance, patient tolerization, etc.) based on the result of the cytokine expression as a function of time is utilized.

In yet another aspect, the invention provides a method for assessing the pharmacodynamic impact (physiological effect) of an immunosupressant drug in a non-transplant patient. The method includes the steps of determining a value of an immune response in at least one sample of lymphocytes from the non-transplant patient; comparing the value with values in a reference set comprising ranges of values of immunological response for lymphocytes; and assessing the pharmacodynamic impact of the immunosupressant drug based on a comparison made in said comparing step. The non-transplant patient may be receiving the immunosupressant drug for a disease condition such as, but not limited to, autoimmunity, inflammation, Crohn's Disease, lupus erythromatosus, or rheumatoid arthritis. The method will typically be carried out in order to reduce complications from infections or cancer in the non-transplant patient.

The present invention provides methods of determining and/or monitoring the state of a subject's immune system. The methods involve measuring lymphocyte activity using a cytokine kinetics assay of the present invention as a measure of immune response, and assignment of the immune response to a standardized range or zone of immune reactivity. The practice of the method thus provides monitoring an individual's immune response at several time points, making possible the characterization of a complete picture of the immune system's reactivity over time. The methods of the present invention make it possible to observe, for example, the response of a patient's immune system to a medical procedure and to adjust treatment protocols accordingly. In addition, using the methods of the present invention, it is possible to predict certain clinical outcomes related to immune system functioning. For example, in a preferred embodiment of the invention, a patient whose immune system is monitored may be on an immunosuppressive drug therapy regimen. In some embodiments, the immunosuppressive drug(s) are administered as the result of an organ transplant. By monitoring the immune response of such a patient, it is possible to predict a risk of rejection of the transplanted organ; or to ascertain if the patient is overmedicated, a condition which can contribute to an increased risk of opportunistic infection, organ toxicity or cardiovascular complication.

It is contemplated that characterization of an individual's immune system response at several time points using a kinetics assay of the present invention permits one to monitor the impact of a course of events on an individual's immune system. For example, a kinetics assay may characterize lymphocytes in a graft recipient before, during and after drug therapy, or before and after organ transplant surgery is performed, in order to monitor changes over time in the immune response of the patient in response to these medical procedures. This information regarding the patient's immune status may be useful as an adjunct to therapeutic drug monitoring at any point in the course of therapy in order to assess the progress of a patient, the suitability of a drug regimen, and to predict clinical outcomes for a patient.

In some embodiments, the present invention provides methods of determining and monitoring the state of a patient's immune system in response to a stimulus. In some embodiments, the patient is one who is receiving or will be receiving an immunomodulating drug or drugs. For example, the patient may be the recipient of an organ such as heart, lung, kidney, pancreas, liver, bowel, skin, bone marrow or other organs. Further, a transplant patient may be the recipient of more than one organ, e.g. a “heart-lung” transplant recipient. Alternatively, the transplant may be transplanted tissue. The transplanted tissue or organ(s) may be from any source known to those of skill in the art, for example, from a live organ donor such as a relative (e.g. a sibling) or a matched or mis-matched non-related donor; from a cadaver; or from a tissue or artificial “organ” that has been developed and/or maintained in a laboratory setting, e.g. tissue or “organs” grown from stem cells, or cultured in a laboratory setting from tissue or cell samples. Alternatively, the patient may be under treatment for an autoimmune disease such as rheumatoid arthritis, lupus, Crohn's disease, psoriasis, etc. Or the patient may be afflicted with an infectious disease, such as Human Immune Deficiency Syndrome related viruses (HIV-1), or Hepatitis associated viruses (HCV). Further, the patient may be a cancer patient. Those of skill in the art will recognize that the methods of the present invention may be used to monitor and/or assess the immune system of any individual for any reason.

In yet another aspect, the invention provides a method for assessing the pharmacodynamic impact of an immunosupressant drug in a non-transplant patient. The method includes the steps of determining (e.g., using a cytokine kinetics assay) a value of an immune response in at least one sample of lymphocytes from the non-transplant patient; comparing the value with values in a reference set comprising ranges of values of immunological response for lymphocytes; and assessing the pharmacodynamic impact of the immunosupressant drug based on a comparison made in said comparing step. The non-transplant patient may be receiving the immunosupressant drug for a disease condition such as autoimmunity, inflammation, Crohn's Disease, lupus erythromatosus, or rheumatoid arthritis.

Those of skill in the art will recognize that many types of immunosuppressive drugs exist that may be administered, the effects of which on the immune system of a patient may be monitored by the methods of the present invention. Examples include but are not limited to antilymphocyte drugs such as OKT3, Antithymocytegamma globulin (ATGAM), Daclizumab, and Basiliximab (anti IL2R); calcineurin inhibitors such as Tacrolimus (PROGRAF, FK506) or cyclosporin (NEORAL); antimetabolites such as Azathioprine, Cyclophosphamide, and Mycophenolate mofetil; enzyme inhibitors such as Sirolimus (Rapamune), or corticocorticoids such as Prednisone, or methylprednisolone (Solumedrol), as well as to drugs currently under investigation in clinical trials. In some embodiments, methods of the present invention (e.g., a cytokine kinetics assay) are utilized to characterize a test compound for use as an immunosuppressive.

The present invention provides a method of guiding decisions regarding therapies and of predicting a clinical outcome of a patient receiving one or more immunosuppressive drugs. Possible clinical outcomes include, for example, rejection of the transplanted organ, infection, or organ toxicity. In order to predict clinical outcomes such as these, it is advantageous to determine an initial lymphocyte functional status as early in the immunosuppressive drug course as possible in order to start surveillance of the patient's immune status coincident with or soon after transplant surgery, but monitoring may begin at any point after the administration of the immunomodulating drugs. Subsequent lymphocyte functional status is ascertained and compared to the earlier response, and to each other.

In a preferred embodiment, an initial sample (e.g., blood ) is obtained and tested (e.g., using a cytokine kinetics assay) prior to transplant (e.g., organ ) surgery and before any immunosuppressant drug is administered. The lymphocyte functional status in the graft recipient is ascertained and compared to established categories of known value ranges (e.g., low, moderate or strong). Based on these values the initial drug dose may be maintained within or modified from the usual practice of dose assignment on the basis of patient body weight. For example, a transplant candidate who is determined to be immunosuppressed due to an infectious disease (e.g. AIDS) may be given a lower or no drug dose, compared to another individual of the same body weight.

In another preferred embodiment, an initial sample (e.g., blood) is obtained and tested (e.g., using a cytokine kinetics assay) prior to organ transplant surgery and before any immunosuppressant drug is administered, and another sample (e.g., blood) is tested (e.g., using a cytokine kinetics assay) after surgery and after the administration of drugs. By comparing the values obtained from these samples, medical judgments can be made relative to the effect of the surgery and drugs on the patient specifically regarding the immune status. For example, if the results indicate that tolerance has been achieved after transplant, the patient may be weaned off of or administered a lower dose of immunosuppressant.

Regarding the frequency at which blood samples are analyzed, those of skill in the art will recognize that sampling may be done at any point at which a skilled practitioner (e.g. a physician) deems it to be advisable. In general, such testing would be carried out at most daily (e.g. during a time when a patient is most at risk) and at least monthly, bimonthly, bi-annually, annually, or longer (e.g. during a time when a patient appears to be relatively stable).

In yet another preferred embodiment, multiple samples are obtained and tested at multiple points after the transplant surgery and during the period when immunomodulating drugs are being administered. An example of the predictive value of the methods would be the detection, by utilizing the methods of the present invention, of an increase in the immune response of the patient (e.g., increased alloreactivity (e.g., kinetically, as opposed to quantifiably (e.g., expression levels) of T cells. The results may be predictive of potential acute rejection of the transplanted organ or tissue, and may warrant, for example: initiation of other confirmatory tests (e.g. organ biopsy or organ specific blood chemistry analyses); an increase in the dose of the drug being administered; a rescue therapy with an alternate drug; or a new combination of drugs.

The method may further be useful for monitoring a patient's immune response during the standard immunosupressive-therapy phase of “weaning” the patient from the drugs (i.e. the phase during which a patient's drug dosage is lowered as much as possible to reduce the risk of toxicity, while maintaining a low chance of transplant rejection). In particular this assay is especially valuable for monitoring tolerance protocols where the objective is the eventual removal of all immunosuppressive drugs. Similarly, the method may also be used to assess patient compliance with prescribed medication regimens.

The method is also of value in monitoring the functional status of the immune responses of long-term organ recipients, who have been on the same medication dosages for extended time periods (years). Patients who have taken immunosuppressive drugs over a long period have been shown to suffer from over suppression concurrent with extended drug courses.

The methods (e.g., cytokine kinetics assays) of the present invention may be used alone as the primary means of tracking a patient's progress. Alternatively, the methods can be used in conjunction with and as an adjunct to other means of assessing a patient's immune status.

In some embodiments, the present invention provides kits comprising reagents for use in cytokine kinetic assays. Various methods can be used to detect cytokines (IFN-γ) including, but not limited to, those described in U.S. Pat. App. 20030215886, herein incorporated by reference in its entirety for all purposes.

In some embodiments, the present invention provides drug-screening assays (e.g., to screen for anti-rejection drugs). The screening methods of the present invention utilize cytokine kinetic assays. For example, in some embodiments, the present invention provides methods of screening for compounds (e.g., “test compounds”) that alter (e.g., increase or decrease) lymphocyte functional status in a subject (e.g., a subject that has undergone organ transplant).

In some embodiments, drug screening assays are performed in animals. Any suitable animal may be used including, but not limited to, baboons, rhesus or other monkeys, mice, or rats. Animals models of transplant rejection are generated (e.g., by performing kidney or other organ transplants or skin transplants on the animals or by the administration of compounds that trigger rejection) and the effect of test compounds on the animal's lymphocyte (e.g., T cell) functional status measured.

Techniques are well known to one of ordinary skill in the art for the transplantation of numerous cell, tissue, and organ types including, but not limited to: pancreatic islet transplantation, corneal transplantation, bone marrow transplantation, stem cell transplantation, skin graft transplantation, skeletal muscle transplantation, aortic and aortic valve transplantation, and vascularized organ transplantation including, but not limited to: heart, lung, heart and lung, kidney, liver, pancreas, and small bowel transplantation (See, e.g., Experimental Transplantation Models in Small Animals (1995) Publisher T&F STM, 494 pages). The present invention is not limited by the particular variety of transplantation.

In general, transplantation between a non-syngeneic donor and recipient, in the absence of a transplant rejection inhibitor results in transplant rejection characterized by the partial or complete, typically progressive, destruction of the transplanted cells, tissue, or organ(s). Accordingly, any non-syngeneic (e.g., allogeneic or xenogeneic) transplantation is useful herein as a model system of transplant rejection. A preferred model system of transplant rejection inhibition comprises a murine allogeneic skin graft.

In some embodiments, non-syngeneic transplants are performed on two groups of mammals, wherein a first group is not treated (the control group) with a test compound (e.g., a transplant rejection inhibitor) and a second group is treated with a test compound. The progress of the transplants are monitored over time utilizing a cytokine kinetics assay of the present invention. In some embodiments, test compounds are identified that increase tolerance within a graft recipient.

Experimental

The following examples are provided in order to demonstrate and further illustrate certain preferred embodiments and aspects of the present invention and are not to be construed as limiting the scope thereof.

In the experimental disclosure that follows, the following abbreviations apply: M (Molar); μM (micromolar); N (Normal); mol (moles); mmol (millimoles); μmol (micromoles); nmol (nanomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); CsA, Cycloprorine A; mAb, Monoclonal antibody; APC, Antigen presenting cell; MLR, Mixed lymphocytes reaction; ELISPOT, Enzyme linked immunospot; CFSE, Carbocyfluorescein diacetate succinimidyl ester; PMA, Phorbol 12-myristate 13-acetate; CTLA4Ig, Cytotoxic T-lymphocyte antigen 4 immunoglobulin;

### EXAMPLE 1

### Materials and Methods

Animals. Male Balb/c (H-2_{d}), C57BL/6J (H-2_{b}) and C3H (H-2ₖ) mice, 6-8 weeks of age, were purchased from Harlan Sprague-Dawley, Inc. (Indianapolis, Ind.). Mice were housed in plastic cages with controlled light/dark cycles and provided ad libitum with food and water. All mouse experiments were performed in accordance with NIH guidelines and in compliance of the University of Wisconsin Laboratory Animal Care and Use Committee.

Skin Transplantation. Full-thickness skin (˜1.5 cm diameter) derived from Balb/c or C57BL/6J donor mice was transplanted on the right dorsal area of C57BL/6J recipient and secured with a plastic adhesive bandage for 7 days. Graft survival was evaluated by daily visual inspection. Necrosis of greater than or equal to 50% of the transplanted skin surface was considered as rejection. Four groups of mice underwent skin transplantation: untreated syngeneic group (n=6), untreated allogeneic group (n=12), allogeneic group treated with daily intraperitoneal injection of 20 mg/kg cyclosporine (n=12), and allogeneic group (n=6) treated with a combination of 500 μg of hamster anti-mouse CD40L monoclonal antibody (mAb; MR1; eBioscience, San Diego, Calif.), 500 μg of human CTLA4-Ig (Chimerigen, Allston, Mass.) and 250 μg of anti-CD25 mAb (PC61, eBioscience). Antibodies were administered intraperitoneally on the day of transplantation (day 0) and on postoperative days 2, 4, and 6.

Histology. All skin grafts were harvested after killing the recipient mouse at the time of rejection, or at 40 days after transplantation in tolerant mice (day 40 after transplantation was set as the end point for graft observation). Skin tissue was fixed in Bouin's solution, embedded in paraffin, cut into 5 μm of sections and stained with hematoxylin and eosin.

IFN-γ kinetics assay. Mouse spleens were harvested and placed into single cell suspension in RPMI 1640 (Life Technologies, Grand Island, N.Y.) by passing through a cell strainer (Becton Dickinson Labware, Franklin Lakes, N.J.). Mononuclear cells were isolated via density gradient centrifugation using lymphocyte separation medium (Mediatech Inc, Herndon, Va.) according to the manufacturer's instructions. After washing twice, cells were adjusted to a concentration of 4×10⁶/ml with culture medium (RPMI 1640 supplemented with 20 mM HEPES, 10 mM sodium pyruvate, 2 mM L-glutamine, 1× MEM-vitamin solution and 15% fetal bovine serum). Then 100 μl of responder C57BL/6J mouse cells were added into a U-bottom 96-well plate (Coming, Corning, N.Y.), and were mixed separately with the same number of irradiated (2000 rad) stimulator cells of C57BL/6J (syngeneic), Balb/c (donor), or C3H (third party) mouse. Each combination was performed in 15 replicates. Cells were incubated at 37° C. in a humidified atmosphere containing 5% CO₂. Starting from day 1 (1 day after the initiation of the culture), 150 μl of culture supernatant was harvested from each well, and 3 wells per day until day 5. The concentration of IFN-γ in the culture supernatant was determined by enzyme linked immunosorbent assay (ELISA) using a kit purchased from R&D systems (Minneapolis, Minn.) according to the manufacturer's instructions. The IFN-γ level for each time point was an average of the measurements of three wells.

IFN-γ enzyme-linked immunospot assay. Allospecific T cell responses were measured by an IFN-γ ELISPOT assay using splenocytes as responder cells obtained from skin-grafted C57BL/6J mice. Anti-mouse IFN-γ monoclonal antibody (BD Bioscience, San Diego, Calif.) was incubated at 5 μg/ml in PBS (100 μl/well) at 4° C. overnight in Unifilter 96 well plates (Whatman, Clifton, N.J.). Following washing with PBS, 4×10⁵ C57BL/6J mouse spleen cells were added to each well of the plate in triplicate. The same number of irradiated (2000 rad) Balb/c donor cells, C3H (third-party) or C57BL/6J (autologous control) mouse spleen cells were added. Cells were incubated for 48 hours at 37° C. in a humidified atmosphere containing 7% CO₂ and 93% N₂. After the coculture, non-adherent cells were removed by washing the plate with PBS containing 0.05% Tween 20. Biotinylated anti-mouse IFN-γ (BD Biosciences) was added at a final concentration of 2 μg/ml (100 μl/well), and the plate was incubated at 4° C. overnight. After washing the plate to remove unbound antibody, Streptavidin-alkaline phosphatase (R&D systems) was added. Spots were visualized with the BCIP/NBT chromogen (R&D systems). Each spot represented an IFN-γ secreting cell, and the spots were enumerated using an ImmunoSpot analyzer (AID, Strassberg, Germany).

T cell proliferation assay using carbocyfluorescein diacetate succinimidyl ester staining. Ten million splenocytes obtained from skin-grafted C57BL/6J mice were resuspended in 1 ml of PBS containing 10 μM of carbocyfluorescein diacetate succinimidyl ester (CFSE) purchased from Molecular Probes (Eugene, Oreg.) and incubated at 37° C. for 10 min. The staining process was then stopped by adding ice-cold Fetal Calf Serum. After three washes with culture medium, 4×10⁵ CFSE-stained responder cells were mixed separately with the same number of irradiated (2000 rad) stimulator splenocytes of C57BL/6J, Balb/c, or C3H mouse. After 4 days culture at 37° C. in a humidified atmosphere containing 5% CO₂, cells were harvested and stained with a rat IgG2a-APC as negative control and APC-conjugated mAbs directed at mouse CD3, CD4, or CD8 (PharMingen, San Diego, Calif.). Flow cytometry was performed using a FACSCalibur (BD Immunocytometry Systems, San Jose, Calif.), and proliferation of responder T cells was analyzed using FlowJo software (Tree Star, Ashland, Oreg.). Percentage of proliferating cells was calculated by proliferating cells (CFSE_{low} cells)/proliferating cells (CFSE_{low} cells)+non-proliferating cells (CFSE_{high} cells) (See, e.g., Hu et al., Transplantation 2003:75: 1075-1077).

Intracellular IFN-γ staining. Intracellular staining for IFN-γ was conducted using a Cytofix/Cytoperm kit (BD Pharmingen) following the manufacturer's instruction. Freshly isolated lymphocytes from naive or rejected mice were activated with phorbol 12-myristate 13-acetate (PMA; 10 ng/ml) and ionomycin (0.4 μg/ml) for 4 hours. Cells were then cultured at 37° C. for 4 hours with the addition of Golgi-stop solution (BD Bioscience) after washing three times with culture medium. For antigen-specific stimulation, responder cells were cultured with irradiated donor cells for 3 days, and Golgi-stop solution (BD Bioscience) was then added into the culture followed by 4 hours incubation prior to the harvest. Cultured cells from the above-described experiments were stained with anti-CD3-Fluorescein (FITC), and anti-NK1.1-allophycocyanin (APC; BD Pharmingen). After permeabilization cells were stained with Phycoerythin (PE)-conjugated mAb directed at IFN-γ. Four-color flow cytometry was performed on a FACScaliber bench-top analyzer (BD Biosciences).

Statistical analysis. Experimental results are presented as mean±standard deviation (SD) or standard error (SE). Multiple comparison test was used to analyze the difference among the groups of experiments by using a computer software GraphPad Prism (GraphPad Software, San Diego, Calif.). To determine if there was a difference in the third-party and donor response of CD3⁺, CD4⁺, and CD8⁺ cells, a paired t-test (SAS software, SAS Institute, Cary, N.C.) was used. A p value less than 0.05 was considered to be statistically significant, unless indicated otherwise herein.

Patients and Protocol Therapy. Primary renal allograft patients received a non-HLA-identical kidney from either cadaveric or living donors. Subjects were 18-60 years of age and had between 1-6 MHC antigen mismatches with the kidney allograft. Kidney transplant recipients were given CAMPATH-1H (Alemtuzumab, ILEX Oncology, Inc.) at day −1 and day 0 of transplant (40 mg total dose), followed by long-term treatment with rapamycin at levels averaging 9 ng/ml. A total of 29 patients were enrolled in the CAMPATH arm of the study. Another 20 patients were enrolled in the control arm. The latter group received anti-CD25 (Basiliximab, Novartis, East Hanover, N.J.) induction therapy, along with conventional long-term immunosuppressants (CsA, steroids, and MMF). All patients gave written informed consent to participate in the study. The protocol was given approval by the Institutional Review Board at the University of Wisconsin-Madison.

CFSE-MLR Assay. Blood was collected at 12 months post-transplant, and PBMC's purified by Ficoll gradient separation. Recipient PBMC's were labeled with 5.0 μM CFSE for 10 minutes at 37° C., upon which 1 ml of cold FCS was added to stop further staining. Cells were then washed once in complete media, and used as responder cells in an MLR in which both proliferation and kinetics of cytokine expression were measured in response to irradiated donor, third party, or autologous stimulator PBMC's. The number of class I and class II MHC mismatches between host/donor and host/third party were kept as consistent as possible with the available third parties from which to choose. A total of 2×10⁵ responder and 2×10⁵ stimulator cells/well were added to a 96-well round-bottom plate, in a total of 200 μl/well complete RPMI with 15% FCS. Cultures were set up in quintuplet wells such that the supernatants could be collected every 24 hours during the 5-day MLR. On day 5, T cell proliferation was measured by flow cytometry using PE-conjugated anti-CD3, CD4, and CD8 antibodies. Live cells were gated out by PI, and subsequent PE-positive cells in the lymphocyte gate were analyzed for their loss of CFSE intensity. On average, 4000 live lymphocytes were acquired for analysis using CellQuest software and a FACSCAN flow cytometer. Proliferation was analyzed using FlowJo software (Tree Star, Inc.).

Cytokine Kinetics Test. Cytokine expression levels in the MLR supernatants were measured using a multi-cytokine fluorescent bead detection system (BeadLyte, Upstate, Inc.). Fifty μl of Day 1-5 supernatants were utilized to analyze the cytokines: IL-1β, IL-2, IL-4, IL-6, IL-8, IL-10, IL-12, GM-CSF, TNF-α, and IFN-γ. Fluorescence was measured using Luminex X-MAP technology (Qiagen, Inc.) CFSE-labeled cells in the culture did not interfere with the detection of any of the cytokines examined.

### EXAMPLE 2

### Costimulation Blockade But Not Cyclosporine Induces Acceptance of Skin Grafts

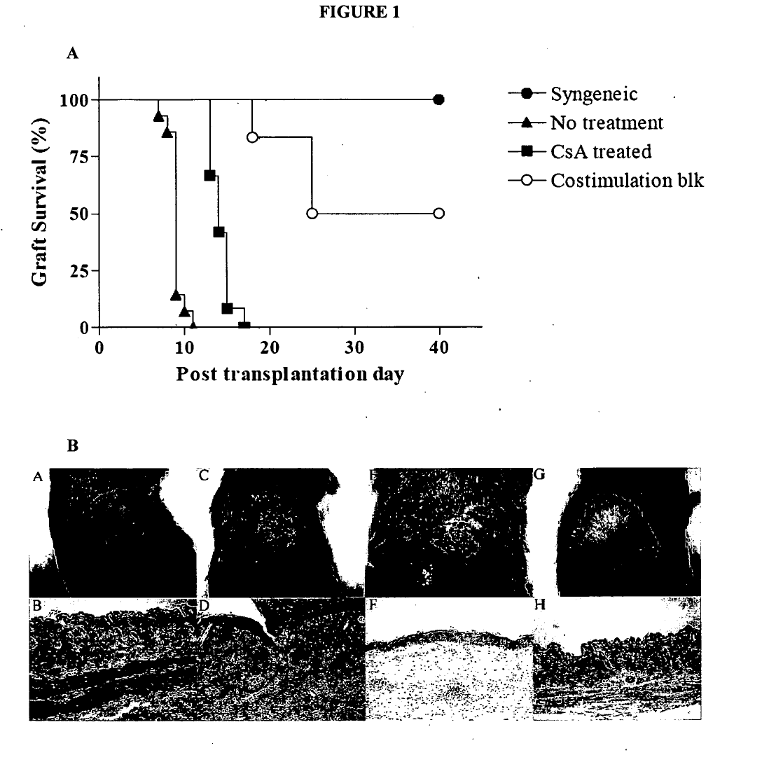

A mouse skin graft model was used to study five categories of immune responses to the grafts. C57BL/6J mice were chosen as recipients and Balb/c mouse as donors. Six syngeneic skin grafts were accepted for at least 40 days of observation, and all 12 allogeneic skin grafts without treatment were rejected within 12 days with an average survival time of 9 days (See FIG. 1). Treatment with CsA (20 mg/kg) significantly prolonged survival of untreated allogeneic transplants (See FIG. 1, MST=14 days, P<0.05) but none of these skin grafts survived more than 17 days. Treatment with four doses of CTLA4-Ig, anti-CD40L mAb and anti-CD25 mAb significantly (P<0.01) prolonged graft survival in 6 recipients to 18, 24, 24, 40, 40, 40 days (mice were sacrificed on day 40). In summary, five distinct results were observed: 1) unmodified syngeneic transplantation with graft acceptance, 2) allogeneic transplantation with graft rejection, 3) allogeneic transplantation treated with CsA with delayed graft rejection, 4) allogeneic transplantation treated with antibody therapy with delayed graft rejection, and 5) allogeneic transplantation treated with antibody therapy with graft acceptance (tolerance). These five groups of recipient mice allowed the identification and characterization of the functional status of host T cells, and correlation of the functional status to the skin graft response.

### EXAMPLE 3

### IFN-γ Kinetics Assay Determines the Functional Status of Alloreactive T Cells

Spleen cells derived from non-transplanted naïve C57BL/6J mouse were used in the IFN-γ kinetics assay in order to observe the naïve T cell response. When stimulated by irradiated donor mouse (Balb/c) spleen cells, IFN-γ was barely detectable on day 1 and day 2 after the culture, but rose significantly from day 3 to day 5 (See FIG. 2a). The same IFN-γ kinetics pattern was observed when spleen cells from naïve C57BL/6J mouse were stimulated by the irradiated third-party C3H mouse spleen cells, while autologous stimulation generated non-detectable IFN-γ in the 5 day cultures (See FIG. 2a).

In untreated C57BL/6J recipient mice, the skin graft was rejected, and spleen cells of these mice were used to study the effector/memory T cell response. When recipient mouse spleen cells were stimulated by donor cells, IFN-γ started at a very high level on day 1 of culture, continued to increase by day 2, and peaked on days 3-5 (See FIG. 2b). Meanwhile, the IFN-γkinetics showed a pattern of naïve response in these mice when the stimulator cells were derived from third-party mice, and, once again, autologous stimulation did not induce any detectable IFN-γ secretion (See FIG. 2b). These mice were also observed for 100 days after rejection, and splenocytes obtained for characterization of the memory T cell response.

INF-γ was not detectable 1 day after the culture when recipient cells were stimulated by irradiated Balb/c spleen cells, but increased drastically on day 2 and maintained a higher level thereafter. The IFN-γ kinetics in culture with third party stimulation showed a naïve response, and autologous stimulation failed to induce detectable IFN-γ (See FIG. 2c). Interestingly, in recipient mice treated with CsA that rejected the skin graft, the IFN-γ kinetics showed an effector/memory response to the donor cells, but no response to cells derived from the third-party mouse and autologous stimulation (See FIG. 2e). In skin grafted mice treated with CsA for 7 days after transplantation, a similar pattern was revealed (See FIG. 2f). Thus, in some embodiments, the present invention provides a method of characterizing host (e.g., transplant recipient host) T cell functional status (e.g., by identifying IFN-γ expression level patterns) prior to rejection of a transplanted graft (e.g., skin or organ graft).

Recipient mice treated with combination therapy (e.g., costimulation and IL-2R blockade) showed two characteristic patterns of IFN-γ production. In response to donor, third-party and autologous stimulator cells, the IFN-γ response in mice that rejected the skin graft was similar to that of recipient mice without treatment. Namely, a graft recipient effector/memory T cell response to donor was observed. Mice with allogeneic skin grafts that survived up to 40 days made a good IFN-γ response to third-party cells with a pattern similar to that of the naïve response. No IFN-γ response was observed in these mice upon stimulation by donor or autologous cells (See FIG. 2d).

### EXAMPLE 4

### Frequency of Donor-Specific IFN-γ-Producing Cells Detected by ELISPOT Assay

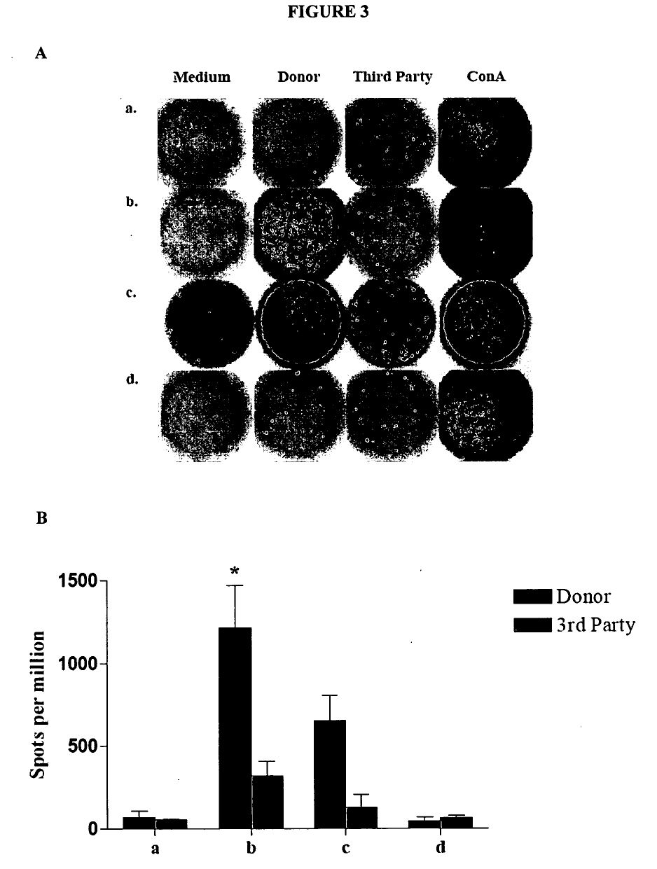

The expression of IFN-γ from the alloreactive T cells of C57BL/6J mouse was also evaluated by ELISPOT assay. Splenocytes from C57BL/6J mice with or without skin grafts were stimulated by irradiated donor (Balb/c) or third-party (C3H) cells, and IFN-γ secretion was evaluated 48 hours after the co-culture. As shown in FIG. 3, incubation with allogeneic stimulator cells (Balb/c) induced a rapid increase of IFN-γ by cells of rejecting mice (effector/memory cells) and by cells from mice that rejected the graft 100 days earlier (memory cells). Responses to third-party stimulation were much weaker. In naïve mice, IFN-γ expression was low in response to both donor and third party stimulation. This low level of IFN-γ secretion also occurred in mice that had prolonged survival of allogeneic skin transplants following costimulation and anti-CD25 blockade.

### EXAMPLE 5

### T Cell Proliferation Determined by CFSE Staining and Flow Cytometry

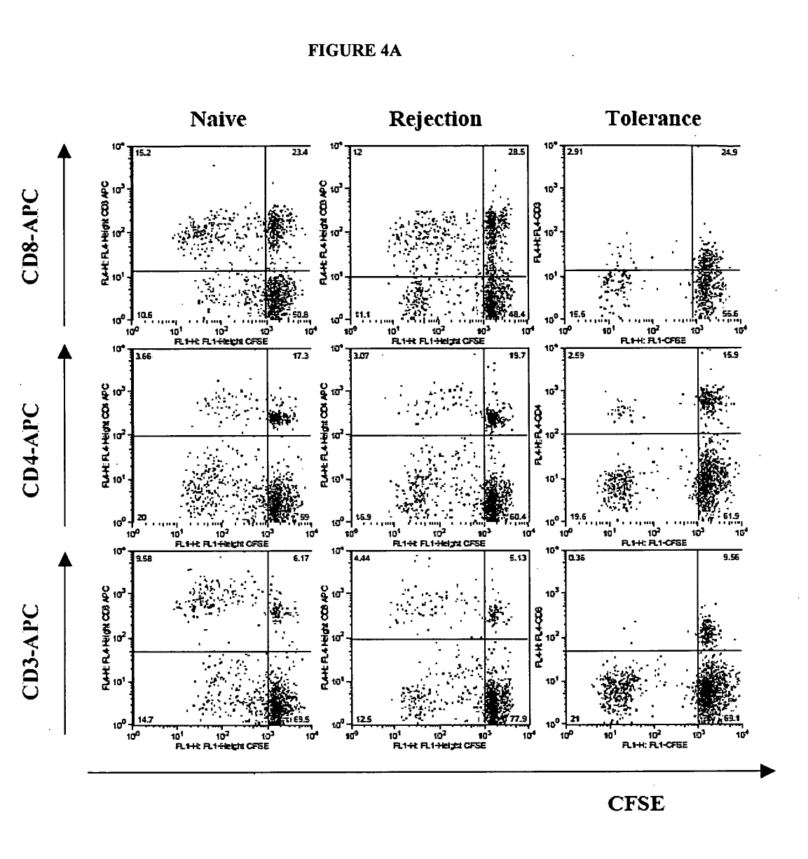

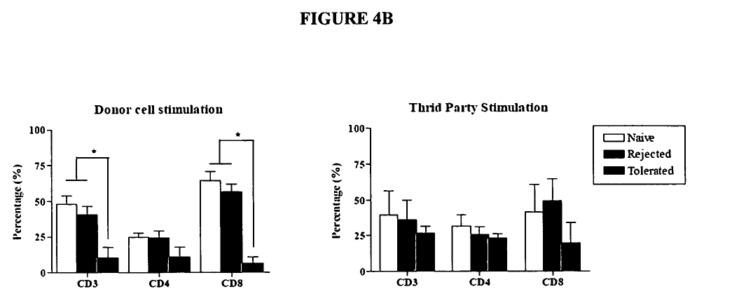

Alloreactive T cell responses were measured by a cell proliferation assay with fluorescent CFSE dye and flow cytometry. Splenocytes from C57BL/6J mice with or without skin grafts were stained with CFSE, and stimulated by irradiated donor (Balb/c) or third-party (C3H) cells. Spleen cells derived from naïve mice and at the time of rejection proliferated comparably in response to donor antigen stimulation (See FIG. 4). This applied to both the CD4+ and CD8+ T cell subsets. CD4+ and CD8+ spleen cells derived from mice 40 days after skin grafting and treatment with costimulation and anti-CD25 blockade proliferated significantly less in response to donor antigen stimulation (P<0.01).

### EXAMPLE 6

### The Cellular Source of IFN-γ in the MLR

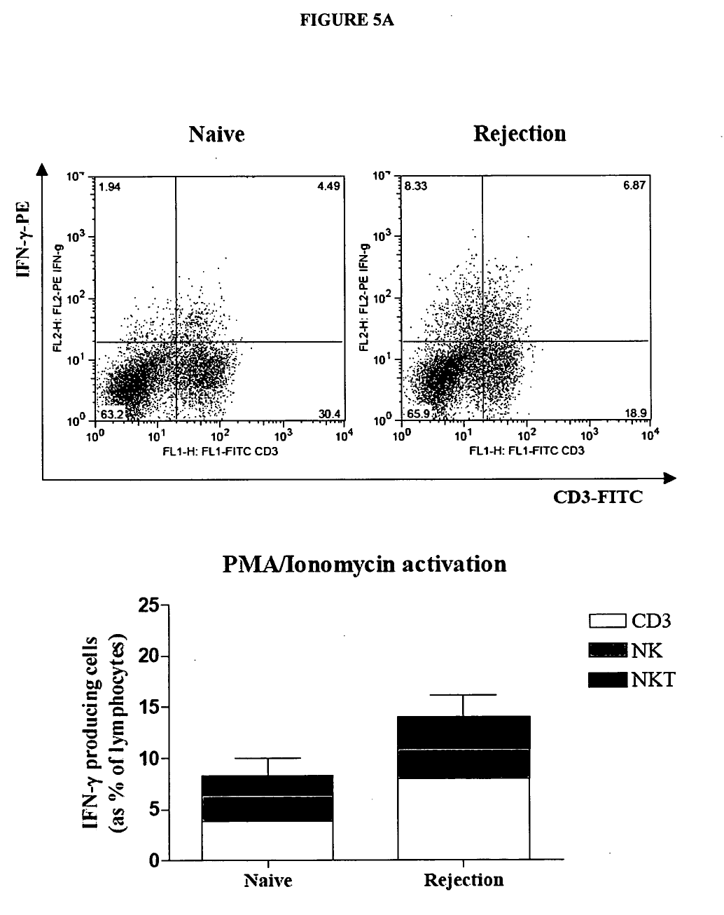

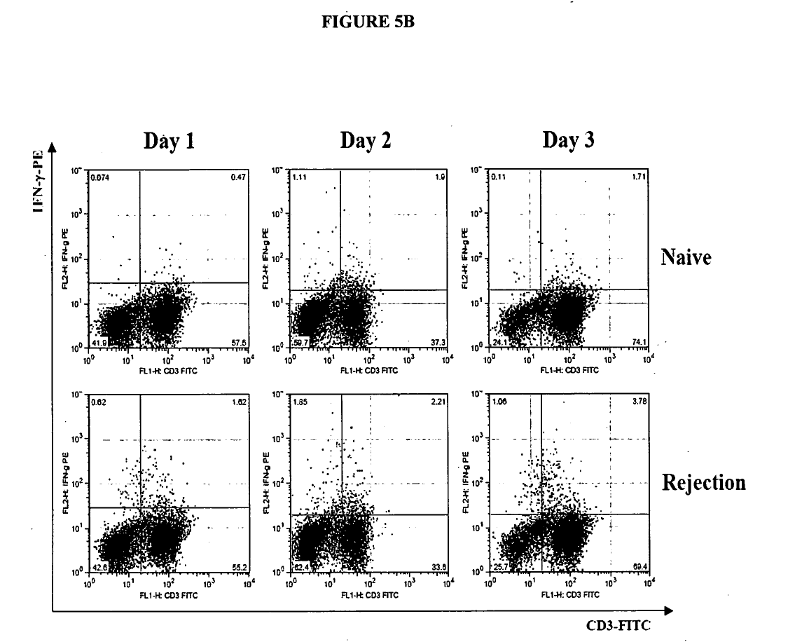

IFN-γ can be produced by many types of cells, including T lymphocytes, NK cells (See, e.g., Yeaman et al., J Immunol 1998:160:5145-5153), and NKT cells (See, e.g., Yoshimoto et al., Proc Natl Acad Sci U S A 1997:94: 3948-3953). In order to confirm that the IFN-γ being measured was produced by responder T cells, intracellular staining and FACS analysis of IFN-γ was performed. IFN-γ expression by T cells, NK cells and NKT cells was assessed in naïve and skin grafted mice with rejection by PMA and ionomycin stimulation (See FIG. 5). PMA/Ionomycin stimulation induces IFN-γ expression from effector/memory T cells but not from naïve T cells (See, e.g., Wang et al., J Immunol 2004:172: 214-221). T, NK, and NKT cells were found to express IFN-γ, while PMA/ionomycin stimulation increased the percentage of IFN-γ producing CD3+ cells in splenocytes derived from skin grafted mice with rejection (See FIG. 5). To confirm that the IFN-γ secreting CD3+ cells in rejecting mice were alloreactive T lymphocytes, IFN-γ production was evaluated after donor cell stimulation. As shown in FIG. 5, IFN-γ production rose faster in T cells derived from rejecting mice than from naïve mice. Thus, the present invention demonstrates that IFN-γ secretion in the kinetic assay and ELISPOT assay derived mainly from alloreactive T cells.

### EXAMPLE 7

### CAMPATH-Treated Patients Have Profound Long-Term T Cell Depletion

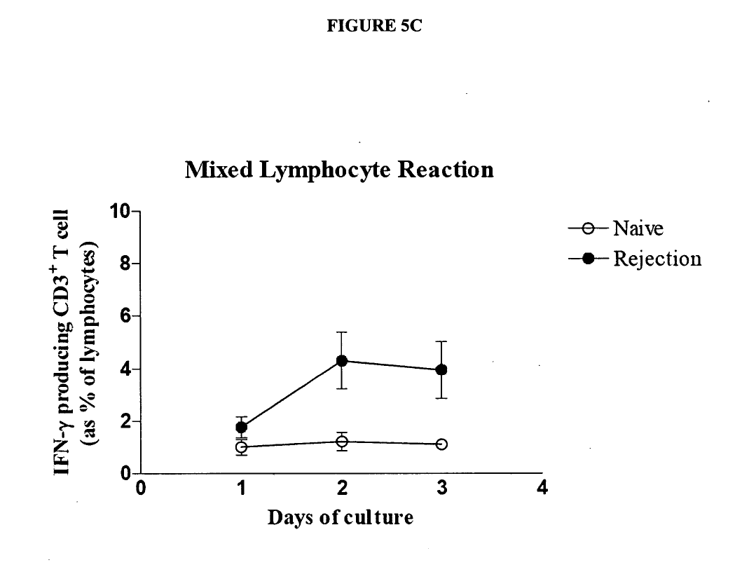

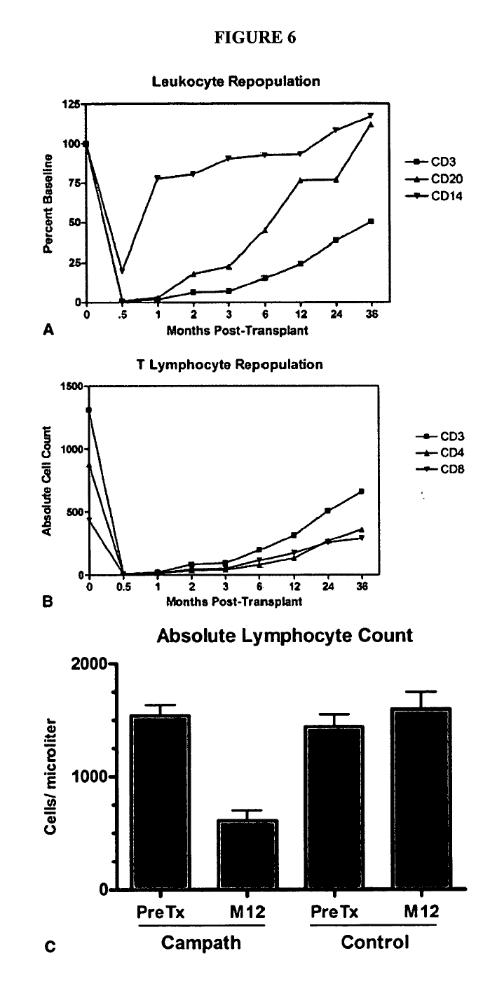

In renal allograft recipients treated with CAMPATH-1H, immunodepletion is profound, with a 2-3 log reduction in peripheral lymphocytes at the outset (See, e.g., Brett et al., Immunology 1996, 88,13). However, depletion of T cells is markedly reduced long-term as well. FIG. 6a shows the mean absolute cell counts for leukocyte subsets in the peripheral blood of 29 CAMPATH patients at multiple time-points over a 3-year period as determined by flow cytometry. Although monocytes and B cells recovered to baseline numbers by 3 and 12-months, respectively, T cell levels were slow to repopulate, recovering to approximately 50% baseline by 36 months. CD4+ and CD8+ T cell subsets were at an approximate 2:1 ratio pre-transplant, yet CD8+ T cells recovered at a relatively constant 1:1 ratio with CD4+ cells in CAMPATH-treated patients (See FIG. 6b). Total lymphocyte counts were established at pre-transplant and month 12 time-points for 11 control patients and 26 CAMPATH patients. FIG. 6c displays the averages of these counts. The present invention demonstrates that the lymphocyte repopulation of CAMPATH patients is well below 50% baseline at month 12, whereas the lymphocyte levels of control patients remain relatively unchanged.

It was also determined to what extent the repopulated T lymphocytes were responsive to donor antigen as compared to T lymphocytes of control patients. Since T cells are relatively few in number in the peripheral blood of CAMPATH patients, in vitro assays were utilized that are highly sensitive (e.g., use few cells), and also provide as much information as possible about the response. A direct one-way MLR using CFSE-labeled responder cells, in conjunction with measuring the kinetics of IFN-γ expression was.

### EXAMPLE 8

### Responses of T Cell Subsets to Donor Alloantigen

For MLR analyses, the 12-month timepoint was analyzed, a time when an adequate number of T cells had repopulated in the peripheral blood of CAMPATH patients. To examine the proliferative response of host T cells to donor antigen, purified responder PBMC's were labeled with CFSE. In an effort to keep the APC source constant, only PBMC's from living donors and third parties were used. APC subpopulations vary within PBMCs and splenocytes, and variation of costimulatory ligands (CD80, CD86, CD40) has been shown to skew a response either towards suppression or activation (See, e.g., Zheng et al., J. Immunol. 2004; 172(5): 2778; Sansom et al., Trends Immunol. 2003; 24(6): 314). Since recipients of cadaveric kidneys were a minority of the allograft recipients, and splenocytes would have been the only available APC source, they were not analyzed. Overall, 17 CAMPATH-rapamycin patients and 10 control patients had living donors and were analyzed as described below.

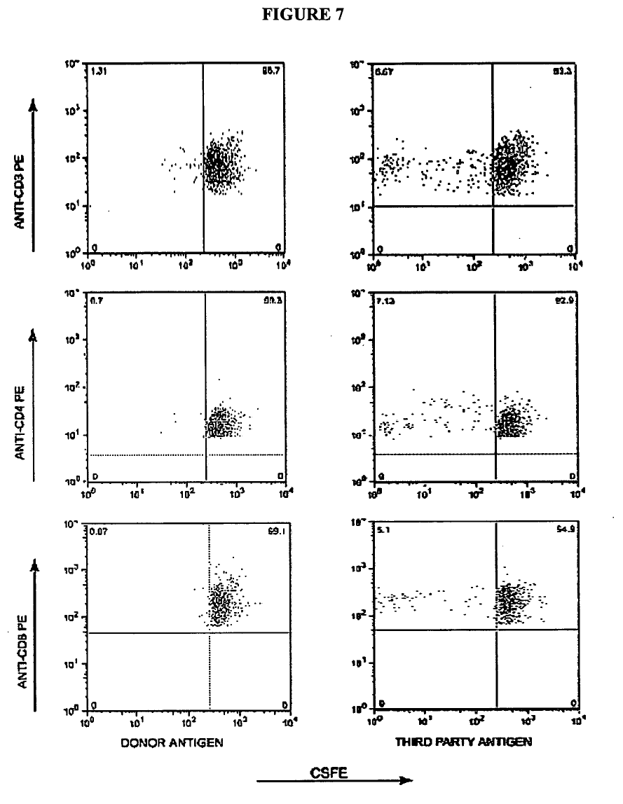

For simplicity, the loss of CFSE intensity was designated “percent proliferation” or “percent CFSE low” (e.g., even though, generally, this is a measure of the actual number of daughter cells initially involved in the response). FIG. 7 displays a representative dot plot series of CFSE proliferation analyses for CAMPATH-treated patient UW19. In response to donor antigen, CD3⁺, CD4⁺, and CD8⁺ cells showed a 1.3, 0.7, and 0.87% shift in CFSE intensity, respectively. Auto-proliferation was typically negligible (<0.5%). In response to third party antigen there was 6.7, 7.1, and 5.1% proliferation of CD3⁺, CD4⁺, and CD8⁺ cells, respectively. The proliferation ratios (% proliferation to donor/% proliferation to third party) are 0.2, 0. 1, and 0.2 for the CD3⁺, CD4⁺, and CD8⁺ populations. Thus, the present invention provides that, by flow analysis alone, this patient was unresponsive to donor antigen via the direct pathway of alloantigen presentation.

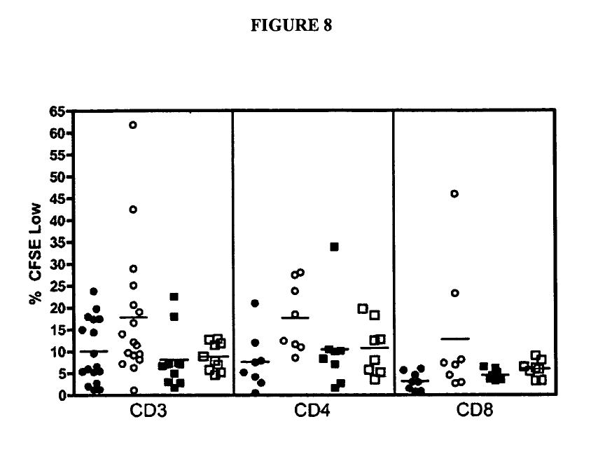

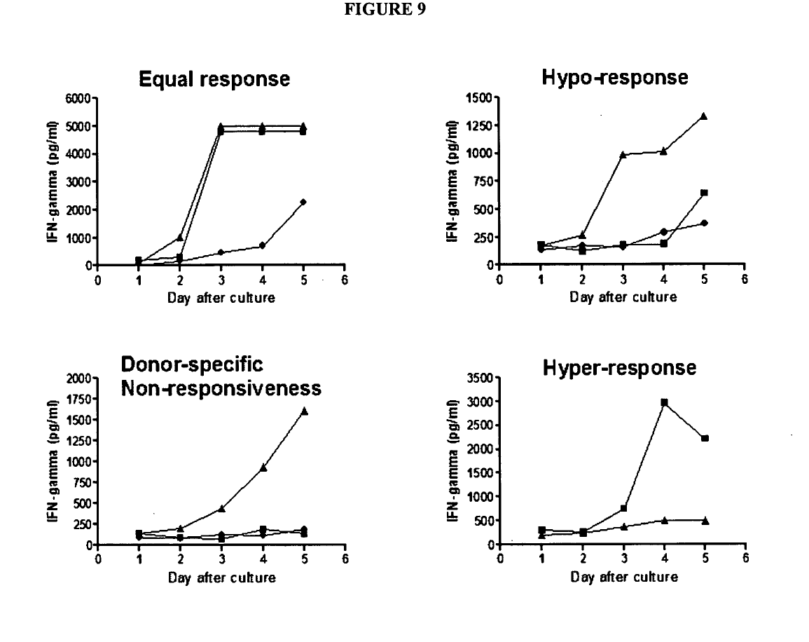

Seventeen renal allograft patients treated with CAMPATH-1H and ten control patients treated with anti-CD25 (Basiliximab) were analyzed by CFSE-MLR. FIG. 8 shows the scatter plot for patients within the CAMPATH and control groups for the percent proliferation to donor versus third party alloantigen for CD3⁺, CD4⁺, and CD8⁺ cells. Statistically, there was no difference in the average percent proliferation to donor antigen between CAMPATH and control groups. However, as a general population, CD3⁺ cells showed a significantly higher response to third party antigen than to donor antigen in the CAMPATH treated patients (P=0.04). The CD8⁺ T cell subset had a similar tendency as the CD3⁺ cells (P<0.01), and the differences in proliferation of CD4⁺ T cells in response to donor and third party antigen approached statistical significance (P=0.07). Conversely, in the anti-CD25 antibody treated recipients, the difference in proliferation between donor and third party responses of CD3⁺, CD4⁺ and CD8⁺ cells had P values that were not statistically significant (0.69, 0.72, and 0.60, respectively).

### EXAMPLE 9

### IFN-γ Kinetics Assay

Additional information was obtained from the 5-day CFSE-MLR by collecting supernatants at 24-hour intervals and analyzing them for the expression of IFN-γ. The kinetic patterning gave a comprehensive picture of alloreactivity which could be categorized into 4 distinct groups: 1) patients who responded equally well to donor and third party antigen, 2) those who were hypo-responsive to donor, 3) those who were hyper-responsive to donor, and 4) those who were completely unresponsive to donor as opposed to third party antigen (See FIG. 4). Of the 15 CAMPATH patients and 8 control patients who were analyzed for IFN-γ expression, the majority of patients (8 CAMPATH, 5 control) could be placed in the hyporesponsive group (See Table 1). Two CAMPATH versus 1 control patient displayed hyper-responsiveness to donor antigen. Statistically, there was no bias in the hypo- (P=0.51) or hyper-responsive (P=0.73) groups using the 1-tailed Fisher's exact test. Four CAMPATH versus zero control patients displayed complete donor-specific non-responsiveness (P=0.15, 1-tailed Fisher's exact test). CD3⁺ T cell proliferation in response to donor antigen was very low or not detectable (<1.8%) in those 4 CAMPATH recipients, yet their absolute T cell counts did not differ significantly from the other CAMPATH patients studied. Therefore, the donor unresponsiveness of these 4 patients cannot simply be attributed to low T cell counts. Taken together, the present invention provides that CAMPATH-rapamycin immunotherapy promotes T cell unresponsiveness in at least a subset of renal allograft recipients.

TABLE 1
Summary of the IFN-γ kinetics assay

| | | Equal | Hypo- | Hyper- | Donor-specific |
|---|---|---|---|---|---|
| Group | n | response | response | response | nonresponsiveness |
| Campath | 15 | 1 | 8 | 2 | 4 |
| Control | 8 | 2 | 5 | 1 | 0 |

### EXAMPLE 10

### Single Drug Maintenance Achieves a Similar Level of Hyporesponsiveness to that of Triple Drug Therapy

Table 2 displays the IFN-γ-kinetics assay results of each CAMPATH and control patient, along with biopsy results and immunosuppressive regimen for that patient. Among the 15 CAMPATH-treated recipients, 10 used a single drug (9 with Sirolimus and 1 with Tacrolimus) for their maintenance immunosuppressive therapy. Of these 10 patients, 7 displayed hyporesponsive or non-responsive T cell alloreactivity to donor antigen. Scheduled protocol kidney biopsies at 12 months after transplantation revealed normal histology in all 10 recipients. In the control group, all 8 patients were treated with a combination of CsA, MMF and prednisone for their immunosuppressive maintenance. Five of these 8 patients revealed T cell hyporesponsiveness to donor antigen stimulation. One patient was biopsied and normal histology was observed. Thus, the present invention demonstrates that CAMPATH patients on maintenance monotherapy have an equal tendency to be hyporesponsive to donor alloantigen as do anti-CD25-treated patients on triple therapy.

TABLE 2
Comparison of clinical outcome and in vitro assays

| | M12 biopsy | Immunosuppression at M12 | IFN-γ assay | GM-CSF assay |
|---|---|---|---|---|
| Campath patients | | | | |
| UW08 | C4d+, humoral rejection | Sirolimus, MMF | HYPO | EQL^{a} |
| UW09 | M06 Biopsy Normal Declined M12 | Sirolimus | EQL | EQL |
| UW10 | Normal | Sirolimus | HYPR | HYPR |
| UW13 | Normal | Sirolimus | UN | UN |
| UW14 | Acute rejection Banff 1A | Tacrolimus, prednisone | UN | UN |
| UW16 | Normal | Sirolimus | HYPO | HYPO |
| UW17 | Normal | Sirolimus | HYPR | HYPR |
| UW19 | Normal | Sirolimus | UN | UN |
| UW21 | Normal | Sirolimus | UN | UN |
| UW22 | Normal | Sirolimus | HYPO | HYPO |
| UW25 | Normal | Sirolimus | HYPO | HYPO |
| UW26 | Normal | Tacrolimus | HYPO | HYPO |
| UW27 | M06 Biopsy normal declined M12 | Tacrolimus, MMF | HYPO | HYPO |
| UW28 | Declined | Tacrolimus, MMF, prednisone | HYPO | HYPO |
| UW29 | Normal | Tacrolimus, MMF | HYPO | HYPO |
| Control Patient | | | | |
| UWC06 | ND | CsA, MMF, prednisone | EQL | EQL |
| UWC08 | ND | CsA, MMF, prednisone | HYPR | HYPR |
| UWC09 | ND | CsA, MMF, prednisone | EQL | EQL |
| UWC11 | ND | CsA, MMF, prednisone | HYPO | EQL |
| UWC14 | ND | CsA, MMF, prednisone | HYPO | HYPO |
| UWC16 | ND | CsA, MMF, prednisone | HYPO | HYPO |
| UWC17 | ND | CsA, MMF, prednisone | HYPO | HYPO |
| UWC20 | ND | CsA, MMF, prednisone | HYPO | HYPO |

^{a}Differences between IFN-γ and GM-CSF readouts.
HYPO, hyporesponsive; EQL, equal response to donor and third-party antigen; HYPR, hyperresponsive to donor Ag; UN, no response to donor Ag; ND, biopsy not done.

### EXAMPLE 11

### Cytokine Multiplex Analysis

To determine whether CAMPATH-derived T lymphocytes stimulated in an MLR were any different in their cytokine expression profiles than those of control patients, the kinetics of expression of IL-2, GM-CSF, IL-4, and IL-10 were also assessed.

IL-2 and IFN-γ expression most closely correlated with T cell proliferation. However, GM-CSF was also a valid indicator of proliferation. GM-CSF expression correlated well with IFN-γ kinetics in 21/23 instances (See Table 2). Thus, like the standard cytokines IL-2 and IFN-γ used to measure reactivity in vitro, the present invention demonstrates that GM-CSF expression can also be used as an indicator of T cell hyporesponsiveness. The non-T cell derived cytokines TNF-α, IL-1β, IL-6, IL-8, and IL-12 were relatively uninformative, as there was no good correlation in expression between T cell proliferation versus hyporesponsiveness in most instances. Levels of these cytokines in the MLR assays ranged from no expression at all (IL-12) to robust expression (IL-8). When assessed for TH2 cytokines, it was determined that IL-4 and IL-10 responses to donor or third party alloantigen were not expressed above levels to self antigen in the MLR's for all CAMPATH and control patients. Thus, none of the mixed lymphocyte reactions were skewed toward a T_{H}2 response.

All publications and patents mentioned in the above specification are herein incorporated by reference. Various modifications and variations of the described methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

What is claimed is:

1. A method for determining the likelihood of transplant rejection in a transplant recipient, comprising
a) providing a sample from said transplant recipient; wherein said sample comprises T cells;
b) exposing said sample to stimulator cells;
c) measuring the level of one or more cytokines expressed by said T cells as a function of time; and
d) correlating cytokine expression as a function of time with the likelihood of transplant rejection.

2. The method of claim 1, wherein said stimulator cells comprise syngeneic antigen presenting cells.

3. The method of claim 1, wherein said stimulator cells comprise allogeneic antigen presenting cells.

4. The method of claim 1, wherein said stimulator cells comprise antigen presenting cells from the transplant donor.

5. The method of claim 1, wherein said method measures IFN-γ.

6. The method of claim 1, wherein said one or more cytokines expressed by said T cells are measured every 24 hours.

7. The method of claim 6, wherein said one or more cytokines expressed by said T cells are measured for three or more days.

8. The method of claim 1, wherein said method identifies a patient that has been tolerized to the transplanted graft.

9. The method of claim 1, wherein said method discriminates between a naïve and memory T cell response in said transplant recipient.

10. The method of claim 1, wherein said patient is receiving one or more immunosuppressive drugs.

11. The method of claim 1, wherein said measuring occurs prior to transplantation.

12. The method of claim 1, wherein said measuring occurs subsequent to transplantation.

13. The method of claim 1, wherein said transplant recipient is selected from the group consisting of a bone marrow transplant recipient, an organ transplant recipient, a tissue transplant recipient and a skin transplant recipient.

14. The method of claim 1, wherein said measuring the level of one or more cytokines comprises detecting nucleic acid sequence.

15. The method of claim 1, wherein said measuring the level of one or more cytokines comprises detecting protein.

16. The method of claim 15, wherein said protein is detected by enzyme linked immunosorbent assay (ELISA).
